(19) United States
(12) Patent Application Publication
(10) Pub. No.: US 2011/0263668 A1
(43) Pub. Date: Oct. 27, 2011

(54) Beta 3-Adrenoreceptor Agonists for the Treatment of Cardiac Hypertrophy and Heart Failure

(75) Inventors: Lili Ayala Barouch, Ellicott City, MD (US); David A. Kass, Columbia, MD (US); An L. Moens, Brasschaat (BG)

(73) Assignee: THE JOHNS HOPKINS UNIVERSITY, Baltimore, MD (US)

(21) Appl. No.: 12/828,642

(22) Filed: Jul. 1, 2010

Related U.S. Application Data
(60) Provisional application No. 61/222,382, filed on Jul. 1, 2009.

(51) Int. Cl. A61K 31/4178; A61K 31/196; A61K 31/404; A61K 31/353; A61P 31/04; A61P 9/12; A61P 3/10; A61P 3/06; A61P 7/04; A61P 7/02; A61K 31/24; A61P 9/00

(52) U.S. Cl. 514/397; 514/539; 514/564; 514/411; 514/456

(57) ABSTRACT

The present invention relates to the field of cardiology. More specifically, the present invention relates to the use of β3 adrenoreceptor agonists to treat cardiac hypertrophy and heart failure. In a specific embodiment, a method for treating cardiac hypertrophy comprises the step of administering a therapeutically effective amount of a β3 adrenoreceptor agonist to a patient diagnosed with cardiac hypertrophy. In a more specific embodiment, the method for treating cardiac hypertrophy comprises the step of administering a therapeutically effective amount of the β3 adrenoreceptor agonist BRL 26830A to a patient diagnosed with cardiac hypertrophy. In a further embodiment, the present invention provides a method for treating a cardiovascular disease or condition associated with cardiac hypertrophy comprising the step of administering a therapeutically effective amount of a β3 adrenoreceptor agonist to a patient diagnosed with cardiac hypertrophy.

### CROSS-REFERENCE TO RELATED APPLICATION

This application claims the benefit under 35 U.S.C. §119(e) of U.S. Provisional Application Ser. No. 61/222,382, filed Jul. 1, 2009, which is entirely incorporated herein by reference.

### STATEMENT OF GOVERNMENTAL INTEREST

This invention was made with U.S. government support under grant no. K08 HL076220, grant no. R01-AG18324, grant no. HL47511, and grant no. P01-HL599408. The U.S. government has certain rights in the invention.

### FIELD OF THE INVENTION

The present invention relates to the field of cardiology. More specifically, the present invention relates to the use of β3 adrenoreceptor agonists to treat cardiac hypertrophy and heart failure.

### BACKGROUND OF THE INVENTION

Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to dilated cardiomyopathy, heart failure, and sudden death. Despite the development and availability of many methods for diagnosis and treatment of cardiac conditions, the morbidity and mortality related to cardiac hypertrophy remains very high.

Heart failure is the most common cause of hospitalization and a leading cause of death in adults over age 55 worldwide (Kass et al. (2009)). In chronic heart failure, the sympathetic nervous system and neuro-hormone are activated, which are initially able to compensate for the depressed myocardial function and preserve cardiovascular homeostasis. However, their long-term activation has deleterious effects on cardiac structure and performance, leading to cardiac decomposition and heart failure progression. Thus, reversing these changes is essential in the treatment of heart failure.

### SUMMARY OF THE INVENTION

The present invention relates to the use of β3 adrenoreceptor agonists to treat cardiac hypertrophy or heart failure. In a specific embodiment, the method for treating cardiac hypertrophy or heart failure comprises the step of administering a therapeutically effective amount of a β3 adrenoreceptor agonist to a patient diagnosed with cardiac hypertrophy or heart failure.

In an alternative embodiment, the method for treating cardiac hypertrophy or heart failure comprises the steps of identifying a patient having cardiac hypertrophy or heart failure; and administering to the patient a therapeutically effective amount of β3 adrenoreceptor agonist.

In a further embodiment, the present invention provides a method for treating a cardiovascular disease or condition associated with cardiac hypertrophy comprising the step of administering a therapeutically effective amount of a β3 adrenoreceptor agonist to a patient diagnosed with cardiac hypertrophy. The cardiovascular disease or condition associated with cardiac hypertrophy may be selected from the group consisting of hypertension, cardiomyopathy, hypertrophic cardiomyopathy, diabetes, systolic heart failure, and non-systolic heart failure.

The β3 adrenoreceptor agonist may be selected from the group consisting of BRL 26830A, SR-58611A (Amibegron), GW-427,353 (Solabegron), L-796,568, CL-316,243, LY-368,842, TAK-677, Ro40-2148, ICI D7114, Carvedilol, and Nebivolol.

In certain embodiments, the methods of the present invention may further comprise administering to the patient a second therapeutic agent. The second therapeutic agent can be selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In yet a further embodiment, the present invention provides a method for treating cardiac hypertrophy or heart failure comprising the step of administering a therapeutically effective amount of the β3 adrenoreceptor agonist BRL 26830A to a patient diagnosed with cardiac hypertrophy or heart failure.

### BRIEF DESCRIPTION OF THE FIGURES

FIG. 1. Cardiac hypertrophy at baseline in the absence of β3-AR. (A) Echocardiographic data showing increased LV wall thickness and mass at baseline in β3^{-/-} compared to age-matched WT at 8 weeks, 4 months, and 14-18 months. Differences are further accentuated with age. (B) Photographic example of old (14-18 months) WT and β3^{-/-} hearts demonstrating increased hypertrophy in β3^{-/-}. *P<0.0001 vs. 8 weeks, ^{†}P<0.05 vs. WT.

FIG. 2. Effect of TAC on mortality and hypertrophy in the absence of β3-AR. (A) Increased mortality in β3^{-/-} mice after mild TAC (P=0.001). (B) Heart weight/tibia length ratio and photographic examples of hearts demonstrating a markedly increased hypertrophic response in β3^{-/-} compared to WT mice with mild TAC. (C) Increased fibrosis in β3^{-/-} TAC vs. WT-TAC hearts as demonstrated by Masson trichrome stain (left panel, 400×) and tabulated using a semi-quantitative scoring system (0=none, 3=marked fibrosis; top right panel). Greater myocyte width in β3^{-/-} TAC vs. WT-TAC by H&E (bottom right). *P<0.05 vs. baseline, ^{†}P<0.01 vs. WT-TAC by post-hoc analysis.

FIG. 3. Effect of TAC on LV dilation and dysfunction in the absence of β3-AR. (A) Examples of M-mode echocardiograms demonstrating decreased systolic function, LV dilation and increased wall thickness in β3^{-/-} TAC vs. WT-TAC. (B) More rapid increase in calculated LV mass in β3^{-/-} TAC vs. WT-TAC by serial echocardiography. (C) Increased LV cavity size by both end-diastolic (LVEDD) and end-systolic (LVESD) dimensions, with concomitant decreases in fractional shortening (FS) and increases in LV wall thickness. (D) Percent changes in wall thickness and calculated LV mass are similar in β3^{-/-} TAC vs. WT-TAC. *P<0.01 vs. β3^{-/-} baseline, ^{†}P<0.01 vs. WT-TAC, ⁵⁵⁵ P<0.05 vs. WT-baseline by post-hoc analysis.

FIG. 4. Changes in NOS activity and NOS isoform expression with TAC. (A) Similar levels of NOS activity after 3 weeks of TAC between β3^{-/-} TAC and WT-TAC by arginine-citrulline conversion. (B) Decreased total NOS activity in β3^{-/-} TAC vs. WT-TAC after 9 weeks of TAC. (C) No differences in eNOS expression levels. (D) Enhanced eNOS activation shown by increased p-eNOS/eNOS ratio in WT-TAC, but no response in β3^{-/-} TAC. (E) nNOS is elevated in β3^{-/-} TAC vs. β3^{-/-} and WT-TAC. (F) iNOS is elevated in β3^{-/-} TAC vs. β3^{-/-} (P<0.01), although no different from WT-TAC. *P<0.05 vs. baseline, ^{†}P<0.05 vs. WT-TAC by post-hoc analysis.

FIG. 5. Elevated NOS-dependent superoxide after TAC in the absence of β3-AR. (A) Increased total superoxide generation in β3^{-/-} TAC vs. β3^{-/-} and WT-TAC. (B) Increased NOS-dependent superoxide in β3^{-/-} TAC vs. WT-TAC calculated by subtracting superoxide production in the presence of L-NAME from total superoxide. (C) Decreased baseline p-Akt/Akt ratio in β3^{-/-}, but no difference between strains after TAC. (D) Reduced GTPCH-1 levels in β3^{-/-} TAC vs. β3^{-/-}. *P<0.05 vs. baseline, ^{†}P<0.01 vs. corresponding WT or WT-TAC by post-hoc analysis.

FIG. 6. BH4 treatment rescues β3^{-/-} mice from pathological hypertrophy and fibrosis. (A) Increased BH4 levels in β3^{-/-} TAC vs. β3^{-/-} (P<0.01), but no difference between strains. (B) Lower BH4/(BH2+Biopterin) ratio in β3^{-/-} hearts compared to WT (P=0.03), but no change with TAC. (C) % change in fractional shortening is lower in β3^{-/-} TAC BH4 vs. β3^{-/-} TAC (P<0.05). (D) % change in LV mass is lower in β3^{-/-} TAC BH4 vs. β3^{-/-} TAC (P<0.01). (E) BH4 treatment lowers NOS-dependent superoxide levels in β3^{-/-} TAC BH4 vs. β3^{-/-} TAC (P<0.05). *P<0.01 vs. baseline, ^{†}P<0.05 vs. corresponding WT or WT-TAC, ^{#}P<0.05 β3^{-/-} TAC BH4 vs. β3^{-/-} TAC by post-hoc analysis.

FIG. 7. Effect of BRL on Left ventricular (LV) dilation and cardiac function in transverse aortic constriction (TAC) mice. (A) Examples of M-mode echocardiograms demonstrating increased LV dilation and wall thickness and decreased systolic function after 3 weeks of TAC. They were improved in mice treated with BRL. (B) BRL restored decreased cardiac function caused by sustained pressure overload back to normal. (C) BRL reduced LV dilation due to TAC. (D) TAC resulted in progressive LV hypertrophy which was partially prevented by BRL treatment. Color coding follows the legend at the top. *P<0.05 vs. sham; †P<0.05 vs. TAC; ‡P<0.05 vs. corresponding 1 week time point.

FIG. 8. Effect of BRL on LV hypertrophy in TAC mice. (A) Increased heart weight to tibia length ratio increased by 3 weeks of TAC was reduced by BRL treatment. This effect was independent of body weight change. (B) Examples of Masson trichrome stain demonstrating increased fibrosis by 3 weeks of TAC (400×). (C) Summary data of increased myocyte diameter and fibrosis scale (semi-quantitative scoring system; 0=none, 3=marked fibrosis) by 3 weeks of TAC. BRL reduced the myocyte diameter while had no change on fibrosis scale. *P<0.05 vs. sham; †P<0.05 vs. TAC

FIG. 9. Changes in superoxide generation by BRL treatment with or without acute nNOS inhibition. (A) 3 week TAC resulted in ˜3.5 fold of lucigenin-enhanced chemiluminescence signal. BRI, significantly reduced this signal. (B) Acute inhibition with nNOS specific inhibitor Vinyl-L-NlO (L-VNlO, 100 uM, 30 minutes in cold room) totally restored the suppressed superoxide generation by BRL treatment back to normal. *P<0.01 vs. sham; †P<0.01 vs. TAC.

FIG. 10. Changes in NOS isoforms protein expression and phosphorylation by BRL treatment in TAC mice. (A) p-eNOS Ser1177/eNOS was decreased and p-eNOS Ser114/eNOS was increased after BRL treatment in TAC mice. (B) p-eNOS Thr495/eNOS and total eNOS expression were unchanged between sham, TAC and TAC-BRL groups. (C) eNOS uncoupling indexed by increased eNOS monomer to dimer ratio was observed after 3 weeks of TAC. BRL treatment did not rescue eNOS uncoupling. (D) nNOS expression was similar between sham and TAC while it was significantly upregulated by BRL treatment. (E) There was a trend toward increase in iNOS expression after 3 weeks of TAC (P=0.06). It was not changed by BRL application. *P<0.01 vs. sham; †P<0.05 vs. TAC.

FIG. 11. Changes in NOS isoform protein expression and phosphorylation by BRL treatment in sham mice. (A) p-eNOS Ser1177/eNOS was similar between sham and sham-BRL while p-eNOS Ser114/eNOS was downregulated after BRL treatment in sham mice. (B) p-eNOS Thr495/eNOS and total eNOS expression were unchanged between sham and sham-BRL. (C) nNOS expression was unchanged by BRL application to sham mice. (D) iNOS protein level was decreased by BRL treatment in sham mice. *P<0.01 vs. sham.

Change of eNOS dimerization by BRL treatment. eNOS uncoupling indexed by increased eNOS monomer to dimer ratio was observed after 3 weeks of TAC. BRL treatment did not rescue eNOS uncoupling. *P<0.05 vs. sham.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

The following definitions are used throughout this specification. Other definitions are embedded within the specification for ease of reference.

The term “β3 Adrenoreceptor Agonist” refers to an agent that stimulates the β3 adrenoreceptor. β3 Adrenoreceptor Agonists are known in the art and include, but are not limited to, BRL 26830A, SR-58611A (Amibegron), GW-427,353 (Solabegron), L-796,568, CL-316,243, LY-368,842, TAK-677, Ro40-2148, ICI D7114, and the like. The term also includes compounds that have activity in addition to β3 Adrenoreceptor Agonistic activity including, but not limited to, Carvedilol and Nebivolol. The term is used interchangeably with “β3 adrenoceptor agonist,” “β3 adrenergic receptor,” “β3-AR” and the like.

The terms “biological sample,” “sample,” “patient sample” and the like, encompass a variety of sample types obtained from an individual and can be used in a diagnostic or monitoring assay. The definition encompasses blood and other liquid samples of biological origin (including, but not limited to, serum, plasma, urine, saliva, stool and synovial fluid), solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as CD4⁻ T lymphocytes, glial cells, macrophages, tumor cells, peripheral blood mononuclear cells (PBMC), and the like. The terms further encompass a clinical sample, and also include cells in culture, cell supernatants, tissue samples, organs, bone marrow, and the like.

As used herein, the term “cardiac hypertrophy” is used in its ordinary meaning as understood by the medical community. It generally refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “effective,” means adequate to accomplish a desired, expected, or intended result. More particularly, a “therapeutically effective amount” as provided herein refers to an amount of a β3 Adrenoreceptor Agonist of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a β3 Adrenoreceptor Agonist, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The terms “heart failure,” “manifestations of heart failure,” “symptoms of heart failure,” and the like are used broadly to encompass all of the sequelae associated with heart failure, such as shortness of breath, pitting edema, an enlarged tender liver, engorged neck veins, pulmonary rales and the like including laboratory findings associated with heart failure.

Optional” or “optionally” means that the subsequently described event or circumstance can or cannot occur, and that the description includes instances where the event or circumstance occurs and instances where it does not.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with cardiac hypertrophy.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causnag regression of the disease, e.g., to completely or partially remove symptoms of the disease.

II. Adverse Ventricular Remodeling and Exacerbated NOS Uncoupling from Pressure-Overload in Mice Lacking the β3-Adrenoreceptor

This study demonstrated that the absence of the β3-AR exacerbates pressure-overload induced NOS uncoupling and subsequent increased NOS-dependent superoxide generation. Consequently, β3^{-/-} mice developed marked adverse remodeling, reflected by increased gross and cellular hypertrophy, fibrosis, LV dilation and depressed LV systolic function.

The upstream regulation of the β3-AR in cardiac myocytes is relatively well established, although its physiological role in the heart and level of interspecies variation remain controversial (Gauthier et al. (2007)). Gauthier et al. demonstrated that β3-AR stimulation decreases cardiac contractility through activation of a NOS pathway (Gauthier et al. (1998)), and studies have suggested this may play a role in cardiodepression observed in cardiac failure and sepsis (Moniotte et al. (2007); Moniotte et al. (2001)). The negative effect is blunted by NOS inhibitors and reversed by an excess of the NOS substrate, 1-arginine (Gauthier et al. (1998)). Imbrogno et al. showed the negative inotropic effect of BRL37344 in isolated hearts from fresh water eels is abolished by exposure to the G_{i/o} inhibitor pertussis toxin (Imbrogno et al. (2006)), and that pre-treatment with inhibitors of soluble guanylate cyclase or cGMP-activated protein kinase G (PKG) abolished β3-AR negative inotropy as well. This supports a central role of Gᵢ-eNOS-NO-cGMP-PKG signaling (Gauthier et al. (1998); Gauthier et al. (1996)). Similarly, mice lacking β3-AR and/or myocytes with the receptor pharmacologically acutely inhibited display enhanced contractile responses to isoproterenol (Morimoto et al. (2004); Varghese et al. (2000)). In vivo, activation of the β3-AR receptor occurs concurrent with β1 and β2 stimulation, so this mechanism can provide a physiologic “brake” to sympathetic over activity.

Removal of this regulatory “brake” in the β3^{-/-} mouse results in an exaggerated response to pressure-overload. The present study supports a major role for NOS as a source of both protective NO and damaging myocardial ROS induced by pressure-overload. At present, it is unknown which NOS isoform is responsible for generating the enhanced levels of ROS in the β3^{-/-} heart. eNOS dysfunction has been demonstrated to play a substantial role in adverse cardiac remodeling, and thus is an attractive candidate (Takimoto et al. (2005)). eNOS normally generates NO to stimulate cGMP and PKG, which protect the heart from hypertrophy and remodeling via transcriptional regulation, phosphorylation, and suppression of targeted signaling, such as from G_{ag} stimulation (Takimoto et al. (2007)). eNOS activity is generally modulated by either translocation or phosphorylation. Phosphorylation at Ser¹¹⁷⁹ (or Ser¹¹⁷⁷ in mouse) activates eNOS, whereas phosphorylation at Thr⁴⁹⁷ or Ser¹¹⁶ is associated with inhibition (Boo et al. (2006)). The increase in eNOS¹¹⁷⁷ phosphorylation seen in WT mice with TAC was blunted in the β3^{-/-} mice, which had no augmentation of eNOS phosphorylation after TAC, indicating an inability of these mice to mount the normal response to pressure-overload. In the normal heart, exposure to severe TAC (≧100% rise in LV mass after 3 weeks) leads to marked eNOS uncoupling, whereas mice lacking eNOS are protected, developing compensated concentric hypertrophy instead (Takimoto et al. (2005)). Others have found that the lack of eNOS exacerbates pathological remodeling if mice are exposed to lower severity banding stress (Ichinose et al. (2004)), perhaps due to less ROS stimulation. In the current study, a milder TAC model was used, as severe pressure-overload proved fatal in all β3^{-/-} mice. Although eNOS remained present, loss of normal NOS activation in the β3^{-/-} mice may have contributed to a ROS/NOS imbalance favoring subsequent NOS uncoupling.

Despite the potential role of eNOS uncoupling, the increases in nNOS and iNOS expression in β3^{-/-} hearts after TAC are intriguing. Both nNOS and iNOS derived NO production have been shown to increase in failing human hearts (Damy et al. (2004); Haywood et al. (1996)), whereas eNOS activity is depressed (Massion et al. (2003)). iNOS may also be cardioprotective in some situations, without causing overt myocyte injury or dysfunction (West et al. (2008)). Following coronary occlusion and reperfusion, iNOS expression in cardiomyocytes was associated with a decrease in oxygen radicals, mitochondrial swelling and permeability transition. Interestingly, β3-AR mediated decrease in cardiac contractility in the diabetic rat heart has shown to be nNOS-dependent (Amour et al. (2007)). Furthermore, nNOS, which is normally localized to the sarcoplasmic reticulum, is found at the sarcolemma after myocardial infarction or in failing hearts, where it serves to decrease β1/2-AR responsiveness in a fashion analogous to β3-AR stimulation (Damy et al. (2004); Damy et al. (2003)). Intriguing recent data produced by Idigo et al. reveals that β3-AR agonist stimulation failed to decrease Ca²⁺ transients and cardiomyocyte shortening in nNOS^{-/-} mice and in WT cardiomyocytes with nNOS inhibition. This was associated with an increase in eNOS-derived superoxide production in nNOS^{-/-} mice, which was abolished by xanthine oxidase inhibition with oxypurinol (Idigo et al. (2006)). This may support a role for nNOS activity in maintaining eNOS coupling by constraining xanthine oxidoreductase activity, with both isoforms potentially acting though β3-AR mediated pathways.

NOS coupling depends upon the bioavailability of the essential NOS cofactor BH4, which in turn depends on expression and activity of the rate-limiting synthetic enzyme GTPCH-1 (Moens et al. (2006)). GTPCH-1 expression decreased in β3^{-/-} TAC in the current study. However, endogenous BH4 levels were not depleted in β3^{-/-} at baseline or following TAC, thereby arguing against this mechanism as being dominant in inducing NOS uncoupling in the β3^{-/-} model. Nevertheless, BH4 treatment did rescue β3^{-/-} mice from adverse remodeling after TAC, with preserved systolic function and lower NOS-dependent superoxide generation. It is unknown whether BH4 requirements increase under conditions of heightened stress to protect against damaging ROS production and maintain NOS coupling. The underlying protective effects of exogenous BH4 may have also been due to direct scavenging of ROS. In addition to decreased BH4 bioavailability, another possible explanation for the exaggerated adverse remodeling is enhanced adrenergic stimulation and consequent ROS generation. Stimulation of β3-AR increases intracellular cGMP, activating PDE2 to enhance its hydrolysis of cAMP (Mongillo et al. (2006)). β3^{-/-} myocardium had blunted eNOS activation, which suppresses cGMP generation (Varghese et al. (2000)), and possibly reducing cAMP hydrolysis by PDE2. Such sustained stimulation can result in calcium mediated injury and myocardial oxidant stress. Lastly, the PI3K/Akt pathway has been proposed as a mechanism for eNOS activation by β3-AR in nonfailing hearts (Brixius et al. (2004)). However, differential Akt activation in β3^{-/-} myocardium subjected to this model of pressure-overload was not observed.

β3-AR are upregulated in human heart failure and animal models (Germack et al. (2006); Moniotte et al. (2001)). Some groups have hypothesized that the negative inotropic effects of β3-AR are detrimental (Gan et al. (2007); Gauthier et al. (2007)), and that diminishing β3-AR activity could be beneficial in the treatment of heart failure (Rozec et al. (2003); Moniotte et al. (2002)). The data presented herein, on the other hand, support the idea that β3-AR serves a chiefly protective role in the heart rather than one depressing contraction, and that blocking this pathway maybe disadvantageous in the stressed or aged myocardium. These data are consistent with the protective effect of β3-AR overexpression reported in a mouse model of isoproterenol-induced heart failure (Belge et al. (2007)).

To the inventors' knowledge, this is the first time that the role of β3-AR in maintaining NOS coupling has been described. Based on these results, it is proposed that β3-AR protects the heart from the long term adverse effects of adrenergic overstimulation, in part by preserving eNOS in its coupled state, despite the fact that acute stimulation of the β3-AR can itself decrease contractility. Additional studies are needed to test the clinical importance of β3-AR in protecting the heart from adverse cardiac remodeling and cardiac hypertrophy.

### III. Cardioprotective Effect of Beta 3 Adrenoreceptor Agonism in Pressure Overload Induced Hypertrophy—The Role of Neuronal NItrix Oxide Synthase

Despite a low level of myocardial expression under basal conditions, accumulating evidence supports a role of up-regulated β3-AR in the modulation of cardiac contraction in heart failure (Amour et al. (2007); Moniotte et al. (2001); and (Gauthier et al. (2000)). Until now direct evidence in vivo has been lacking. As shown herein, the comparison of the cardiac response to pressure overload in both WT and β3^{-/-} mice revealed worse hypertrophy and cardiac systolic function in β3^{-/-} mice than WT controls (Moens et al. (2009)). Also as described herein, β3-AR agonism exerts a cardioprotective role after pressure overload. Administering specific β3-AR agonist BRL to C57BL/6 mice for 3 weeks totally prevented the deterioration of LV chamber dilation and cardiac dysfunction, and partially inhibited myocardial hypertrophy induced by chronic pressure-overload. This strongly suggests that specific β3-AR agonism might constitute an interesting new approach to treating cardiac hypertrophy and heart failure. This beneficial role of β3-AR stimulation was associated with increased NO production and reduced superoxide generation. Moreover, a 2 fold of increase of nNOS protein expression was observed in the BRL treated group, indicates a possible explanation for superoxide suppression by BRL. Importantly, the other two NOS isoforms, eNOS and iNOS were not identified in the β3-AR regulation of cardiac function. Thus, β3-AR activation may cause NO production and reactive oxygen species (ROS) reduction through a nNOS-dependent mechanism in the failing heart.

Role of nNOS in β3-AR Cardioprotection. Studies have identified that the β3-AR-induced negative inotropic effect was associated with NO release via NOS (Varghese et al. (2000); Gauthier et al. (1998)). The current study demonstrated decreased NO production in TAC mice, consistent with the literature (Liao et al. (2004)). Chronic β3-AR stimulation in the model described herein prevented the decrease in NO production during pressure overload, which is consistent with data also described herein that NOS activity is decreased in β3^{-/-} mice after TAC (Moens et al. (2009)). However, which isoform of NOS is involved in β3-AR regulation still remains controversial. Although previous studies assumed that cardiac eNOS was the sole source of NO involved in the regulation of myocardial contraction (Gauthier et al. (1998)), emerging evidence indicates that nNOS derived NO production at least play a part in the regulation of basal and β-AR myocardial contraction (Bendall et al. 2004)). Furthermore, nNOS was up-regulated in senescent rat hearts after myocardial infarction and in human failing hearts (Bendall et al. (2004); Damy et al. (2004); and Damy et al. (2003)). nNOS gene deletion has been associated with more severe LV remodeling and functional deterioration in murine models of myocardial infarction, suggesting that nNOS derived NO may also be involved in the myocardial response to injury (Dawson et al. (2005); Saraiva et al. (2005)). The present study revealed exclusive nNOS activation by β3-AR agonism, which suggested nNOS-derived NO production plays a role in the cardioprotective effect of β3-AR agonism in pressure overload hypertrophy and heart failure.

Recently, it was demonstrated that positive inotropic response to β-AR stimulation was impaired in diabetic and aged rat hearts, and was restored by a β3-AR antagonist, a nonselective NOS inhibitor and the selective nNOS inhibitor L-VNIO (Birenbaum et al. (2008); Amour et al. (2007)). An ex vivo study from ldigo et al. (2006) also showed negative inotropic response to β3-AR agonism BRL in cardiomyocytes was absent in both nNOS^{-/-} cardiomyocytes and WT cardiomyocytes with pharmacological inhibition of nNOS. These studies support nNOS derived NO production a primary factor in altered contractile response by β3-AR stimulation of the heart. The pathway regulating cardiac contractility may be associated with nNOS translocation from sarcoplasmic reticulum (SR) to sarcolemma, where the enzyme interacts with caveolin-3, then impaired the myocardial contractility to isoproterenol (Bendall et al. (2004)).

In the present study, both eNOS and iNOS protein expressions were unchanged by BRL treatment. eNOS activity is generally modulated by either translocation or phosphorylation. eNOS translocation was observed by β3-AR stimulation only in right atrium, not in left ventricle (Brixius et al. (2006); Brixius et al. (2004)). Ser¹¹⁷⁷ and Ser¹¹⁴ are two phosphorylation sites which can modulate eNOS activity. Phosphorylation at Ser¹¹⁷⁷ (or Ser1179 in human) activates eNOS, whereas phosphorylation at Ser¹¹⁴ deactivates eNOS²⁹⁻³¹. A decrease in Ser¹¹⁷⁷ phosphorylation and an increase in Ser¹¹⁴ phosphorylation after BRL treatment was observed, which suggested eNOS deactivation rather than activation by β3-AR stimulation. A recent study from isolated human failing myocardium reported similar results (Napp et al. (2009)). The discrepancy between β3-AR stimulation induced NO-dependent negative inotropic effect and eNOS deactivation in human failing myocardium could be explained by nNOS activation in cardiomyocytes. Paracrine negative inotropic effect via NO liberation from cardiac endothelial cells may be another explanation, but lacking direct evidence until recently. The same group also reported that eNOS was activated through Ser¹¹⁷⁷ phosphorylation by BRL in human non-failing myocardium, which identified different downstream signal of NOS isoform by β3-AR stimulation between failing and nonfailing hearts (Brixius et al. (2006); Brixius et al. (2004)).

Inhibition of Oxidative Stress. A significant number of animal studies and several clinical observations have demonstrated ROS activation in the cardiovascular system in response to various stressors and in the genesis of the hypertrophic and failing heart (Wang et al. (2010); Sheeran et al. (2010); and Sawyer et al. (2002)). Biomarkers for ROS have been detected in the pericardial fluid as well as in the peripheral blood of heart failure patients (Mallat et al. (1998)). Further experiments showed that ROS is up-regulated in β-AR stimulation-induced cardiac hypertrophy and remodeling (Bajcetic et al. (2008); Kawai et al. (2004)). However, the modulation of β3-AR stimulation on ROS generation has not been clearly defined. In the study described herein using β3-AR^{-/-} mice, increased NOS-dependent generation of the reactive oxygen species superoxide was observed, implying that NOS dependent ROS may be one of the downstream signals of β3-AR (Moens et al. (2009)). Further work also described herein confirmed this point by showing that chronic pressure overload induced a marked increase in superoxide generation with substantial reduction by BRL treatment. eNOS was uncoupled by 3 weeks of TAC, indicated by increased eNOS m/d ratio, which is in agreement with previous reports (Moens et al. (2008); Takimoto et al. (2005)). However, eNOS was not re-coupled by BRL treatment as no change of eNOS m/d ratio between BRL treated mice and vehicle, furthering evidence that eNOS may not be the sole downstream NOS signal as previously thought. More importantly, suppression of ROS generation by BRL was abolished by 30 minutes acute inhibition of nNOS by preferential nNOS inhibitor, L-VNIO, at a concentration only inhibits nNOS without affecting other NOS isoforms. These results revealed the antioxidant effect of β3-AR agonism is dependent on nNOS activation, though the underlined mechanism remains unclear. Recently, it was shown that deficiency of nNOS leads to profound increase in xanthine oxidoreductase (XOR)-mediated superoxide production without affecting XOR mRNA or protein abundance, which depresses myocardial excitation-contraction coupling in a manner reversible by XOR inhibitor (Kinugawa et a. (2005); Khan et al. (2004)). This suggests constrained XOR activity by nNOS as a possible connection between myocardial NOS and ROS systems. Thus, the cardioprotective effect of β3-AR agonism on cardiac hypertrophy and heart failure could be attributed to nNOS activation which favors the equilibrium of myocardial NO and ROS production.

Clinical Implication. Heart failure is the most common cause of hospitalization and a leading cause of death in adults over age 55 worldwide (Kass et al. (2009)). In chronic heart failure, the sympathetic nervous system and neuro-hormone are activated, which are initially able to compensate for the depressed myocardial function and preserve cardiovascular homeostasis. However, their long-term activation has deleterious effects on cardiac structure and performance, leading to cardiac decomposition and heart failure progression. Thus, reversing these changes is essential in the treatment of heart failure. β1 blockers have become the standard treatment of chronic heart failure after 1990. It is proposed herein that I33-AR agonism can be thought of as a functional β1 blocker due to its negative inotropic effect on human myocardium. In the current study, the preferential β3-AR agonist BRL is used and it directly showed that in mice subjected to chronic pressure-overload, BRL prevented progressive LV chamber dilation and cardiac dysfunction and inhibited cardiomyocyte hypertrophy. This effect of BRL is linked to increased NO production and reduced oxidative stress by nNOS activation. This study directly and strongly supports the notion that β3-AR plays a beneficial role in heart and highlights the potential therapeutic utility of β3-AR agonist for heart failure and myocardium hypertrophy treatment. Although low expression levels of β3-AR in human tissues have resulted in disappointing outcomes from animal studies to clinical trials evaluating β3-AR agonists for obesity, type 2 diabetes and irritable bowel syndrome treatment (Rasmussen et al. (2009); Arch et al. (2008); Clouse et al. (2007); and Arch et al. (2002)), heart failure may represents a more realistic therapeutic target for β3-AR agonist for three main reasons. First, β3-AR has been demonstrated to be expressed at levels that can mediate physiological responses in healthy human myocardium (Gauthier et al. (1996)). Second, β3-AR is up-regulated 2-3 fold in the progression of heart failure (Moniotte et al. (2001)). Especially with the down-regulation of β1-AR, increased β3: β1-AR ratio likely plays a more substantial role than previously thought. This may also compensate for the bioavailability and selectivity problems of orally administered β3-AR agonists (Bristow et al. (1982)). Lastly, co-treatment with conventional β-blockers can further increase the expression of β3-AR. A study in diabetic rats demonstrated that chronic treatment with metoprolol markedly increased the expression of the cardiac β3-AR (Sharma et al. (2008)). A very recent study reported that the hemodynamic parameters improvement obtained from the third-generation β-blocker nebivolol administration in heart failure patients is partially due to its NO-dependent negative inotropic effect by β3-AR stimulation which is similar to the β3-AR preferential agonist BRL (Rozec et al. (2009)).

In conclusion, β3-specific agonism in vivo has substantial cardioprotective effects, and that these effects may be attributable to nNOS activation. These findings have direct therapeutic implications for treating heart failure patients.

### IV. Pharmaceutical Compositions

A. Formulations

The present invention also provides pharmaceutical compositions. Such compositions comprise a β3 Adrenoreceptor Agonist of the present invention. The composition further comprises a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the β3 Adrenoreceptor Agonist is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a β3 Adrenoreceptor Agonist together with a suitable amount of a pharmaceutically acceptable carrier so as to provide the form for proper administration to the patient. The formulation should suit the mode of administration.

Furthermore, a β3 Adrenoreceptor Agonist of the present invention can be administered with compounds that facilitate uptake of the β3 Adrenoreceptor Agonist by target cells or otherwise enhance transport of an agonist to a particular site for action. Absorption promoters, detergents and chemical irritants (e.g., keratinolytic agents) can enhance transmission of an agonist into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Suitable agents for use in the methods of the present invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

In other embodiments, a colloidal dispersion system may be used for targeted delivery of the β3 Adrenoreceptor Agonist to specific issue. Colloidal dispersion systems include macromolecule complexes, nanocapsules, microspheres, beads, and lipid-based systems including oil-in-water emulsions, micelles, mixed micelles, and liposomes.

B. Routes of Administration

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means.

C. Dosage Determinations

In general, the pharmaceutical compositions disclosed herein may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., a pharmaceutical composition comprising a β3 Adrenoreceptor Agonist in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either were used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53,54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of humans or animals can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about I month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

D. Combination Therapy

It would be readily apparent to one of ordinary skill in the art that the pharmaceutical compositions of the present invention (e.g., the β3 Adrenoreceptor Agonists) can be combined with one or more therapeutic agents. In particular, the compositions of the present invention and other therapeutic agents can be administered simultaneously or sequentially by the same or different routes of administration. The determination of the identity and amount of therapeutic agent(s) for use in the methods of the present invention can be readily made by ordinarily skilled medical practitioners using standard techniques known in the art. In specific embodiments, a β3 Adrenoreceptor Agonist of the present invention can be administered in combination with an effective amount of a therapeutic agent that treats cardiac hypertrophy and/or any heart disease associated with cardiac hypertrophy.

Therapeutic agents include, but are not limited to, beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

More specifically, a β3 Adrenoreceptor Agonist may be combined with a therapeutic including, but not limited to, an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof

In specific embodiments, a β3 Adrenoreceptor Agonist may be combined with an antihyperlipoproteinemic agent including, but not limited to, aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof, acifran, azacosterol, benfluorex, β-benzalbutyramide, camitine, chondroitin sulfate, clomestrone, detaxtran, dextran sulfate sodium, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

A β3 Adrenoreceptor Agonist may be combined with an antiarteriosclerotic agent such as pyridinol carbamate. In other embodiments, a β3 Adrenoreceptor Agonist may be combined with an antithrombotic/fibrinolytic agent including, but not limited to anticoagulants (acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin); anticoagulant antagonists, antiplatelet agents (aspirin, a dextran, dipyridamole (persantin), heparin, sulfinpyranone (anturane) and ticlopidine (ticlid)); thrombolytic agents (tissue plaminogen activator (activase), plasmin, pro-urokinase, urokinase (abbokinase) streptokinase (streptase), anistreplase/APSAC (eminase)); thrombolytic agent antagonists or combinations thereof);

In other embodiments, a β3 Adrenoreceptor Agonist may be combined with a blood coagulant including, but not limited to, thrombolytic agent antagonists (amiocaproic acid (amicar) and tranexamic acid (amstat); antithrombotics (anagrelide, argatroban, cilstazol, daltroban, defibrotide, enoxaparin, fraxiparine, indobufen, lamoparan, ozagrel, picotamide, plafibride, tedelparin, ticlopidine and triflusal); and anticoagulant antagonists (protamine and vitamine K1).

Alternatively, a β3 Adrenoreceptor Agonist may be combined with an antiarrhythmic agent including, but not limited to, Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

Non-limiting examples of sodium channel blockers include Class IA (disppyramide (norpace), procainamide (pronestyl) and quinidine (quinidex)); Class IB (lidocaine (xylocalne), tocamide (tonocard) and mexiletine (mexitil)); and Class IC antiarrhythmic agents. (encamide (enkaid) and flecamide (tambocor)).

Non-limiting examples of a beta blocker (also known as a β-adrenergic blocker, a β-adrenergic antagonist or a Class II antiarrhythmic agent) include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

Non-limiting examples of an agent that prolong repolarization, also known as a Class III antiarrhythmic agent, include amiodarone (cordarone) and sotalol (betapace).

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

In other embodiments, a β3 Adrenoreceptor Agonist may be combined with an antihypertensive agent including, but not limited to, alpha/beta blockers (labetalol (normodyne, trandate)), alpha blockers, anti-angiotensin II agents, sympatholytics, beta blockers, calcium channel blockers, vasodilators and miscellaneous antihypertensives.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

Non-limiting examples of anti-angiotension II agents include angiotensin converting enzyme inhibitors and angiotension II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-I receptor blocker (ARBS), include angiocandesartan, eprosartan, irbesartan, losartan and valsartan.

Non-limiting examples of a sympatholytic include a centrally acting sympatholytic or a peripherially acting sympatholytic. Non-limiting examples of a centrally acting sympatholytic, also known as a central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or an α1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

In certain embodiments a antihypertensive agent may comprise a vasodilator (e.g., a cerebral vasodilator, a coronary vasodilator or a peripheral vasodilator). In particular embodiments, a vasodilator comprises a coronary vasodilator including, but not limited to, amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative (amosulalol, bufuralol, dilevalol, labetalol, pronethalol, sotalol and sulfinalol); a benzothiadiazine derivative (althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlonnethiazide); a N-carboxyalkyl(peptide/lactam) derivative (alacepril, captopril, cilazapril, delapril, enalapril, enalaprilat, fosinopril, lisinopril, moveltipril, perindopril, quinapril and ramipril); a dihydropyridine derivative (amlodipine, felodipine, isradipine, nicardipine, nifedipine, nilvadipine, nisoldipine and nitrendipine); a guanidine derivative (bethanidine, debrisoquin, guanabenz, guanacline, guanadrel, guanazodine, guanethidine, guanfacine, guanochlor, guanoxabenz and guanoxan); a hydrazines/phthalazine (budralazine, cadralazine, dihydralazine, endralazine, hydracarbazine, hydralazine, pheniprazine, pildralazine and todralazine); an imidazole derivative (clonidine, lofexidine, phentolamine, tiamenidine and tolonidine); a quantemary ammonium compound (azamethonium bromide, chlorisondamine chloride, hexamethonium, pentacynium bis(methylsulfate), pentamethonium bromide, pentolinium tartrate, phenactropinium chloride and trimethidinium methosulfate); a reserpine derivative (bietaserpine, deserpidine, rescinnamine, reserpine and syrosingopine); or a suflonamide derivative (ambuside, clopamide, farosemide, indapamide, quinethazone, tripamide and xipamide).

In other embodiments, a β3 Adrenoreceptor Agonist may be combined with a vasopressor. Vasopressors generally are used to increase blood pressure during shock, which may occur during a surgical procedure. Non-limiting examples of a vasopressor, also known as an antihypotensive include amezinium methyl sulfate, angiotensin amide, dimetofrine, dopamine, etifelmin, etilefrin, gepefrine, metaraminol, midodrine, norepinephrine, pholedrine and synephrine.

A β3 Adrenoreceptor Agonist may be combined with treatment agents for congestive heart failure including, but not limited to, anti-angiotension II agents, afterload-preload reduction treatment (hydralazine (apresoline) and isosorbide dinitrate (isordil, sorbitrate)), diuretics, and inotropic agents.

Non-limiting examples of a diuretic include a thiazide or benzothiadiazine derivative (e.g., althiazide, bendroflumethazide, beizthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Alternatively, therapeutic agents that can be administered in combination therapy with one or more β3 Adrenoreceptor Agonists include, but are not limited to, anti-inflammatory, anti-viral, anti-fungal, anti-mycobacterial, antibiotic, amoebicidal, trichomonocidal, analgesic, anti-neoplastic, anti-hypertensives, anti-microbial and/or steroid drugs, to treat cardiac hypertrophy and/or any heart disease associated with cardiac hypertrophy. In some embodiments, patients are treated with a β3 Adrenoreceptor Agonist in combination with one or more of the following; β-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddl, Videx™), stavudine (d4T, Zerit™), zalcitabine (dideoxycytosine, ddC, Hivid™), nevirapine (Viramune™), lamivudine (Epivir™, 3TC), pro tease inhibitors, saquinavir (Invirase™, Fortovase™), ritonavir (Norvir™), nelfinavir (Viracept™), efavirenz (Sustiva™) abacavir (Ziagent™), amprenavir (Agenerase™) indinavir (Crixivan™), ganciclovir, AzDU, delavirdine (Kescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparagyinase and combinations foregoing.

In another aspect, the β3 Adrenoreceptor Agonists of the present invention may be combined with other therapeutic agents including, but not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), β2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), other anti-viral agents, and antibiotics (e.g., dactinomycin (formerly actinomycin), bleomycin, erythomycin, penicillin, mithramycin, and anthramycin (AMC)).

In various embodiments, a β3 Adrenoreceptor Agonist of the present invention in combination with a second therapeutic agent may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, a β3 Adrenoreceptor Agonist of the present invention and one or more other therapies are cyclically administered. Cycling therapy involves the administration of a first therapy (e.g., a β3 Adrenoreceptor Agonist) for a period of time, followed by the administration of a second therapy (e.g. a second β3 Adrenoreceptor Agonist or another therapeutic agent) for a period of time, optionally, followed by the administration of a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Adverse Ventricular Remodeling and Exacerbated NOS Uncoupling from Pressure-Overload in Mice Lacking the β3-Adrenoreceptor

Experimental Model. Baseline echocardiography was performed on 8-week, 4-month, and 14-18 month-old homozygous β3^{-/-} mice (n=57 total, original breeding pairs kindly provided by Dr. Bradford Lowell (Klein et al. (1999)) and age-matched FVB background WT controls (n=13, Jackson Laboratories, Bar Harbor, Me.). Transverse aortic constriction (TAC) was performed on 8-week-old male β3^{-/-} (n=21) and age-matched male WT controls (n=24) as previously described (Takimoto et al. (2005)). Briefly, after anesthesia with isoflurane (2%), the chest was opened through a small thoracic window between ribs 2 and 4, and a 25 G needle placed on the transverse aorta. This needle size was chosen to elicit a milder response as initial studies using a standard TAC model (27 G needle) led to pulmonary edema and 100% early mortality in β3^{-/-} mice. The band was secured using a 7.0 prolene suture, the needle was then removed and the chest closed. Twelve animals per strain underwent sham surgery. To measure pressure changes after TAC, pressure volume loops were obtained using a Millar micromanometer catheter as previously described (Barouch et al. (2002)). Animals were sacrificed 3 or 9 weeks after TAC and myocardial tissue preserved in 10% formalin or snap-frozen in liquid nitrogen for subsequent analysis. To determine whether there were any differences in proximal pressures after TAC between strains, a 1.4 F pressure catheter (Millar Instruments, Houston, Tex.) was advanced into the ascending aorta from the LV, and pressures recorded before and after TAC. Mice were housed in a university animal facility with a 12-hour light-dark cycle and allowed water and food ad libitum. For tetrahydrobiopterin (BH4) treated mice, 200 mg/kg/day (Schircks Laboratories, Jona, Switzerland) or vehicle was mixed in soft diet. Animal treatment and care was provided in accordance with institutional guidelines. The Institutional Animal Care and Use Committee of The Johns Hopkins University School of Medicine approved all protocols and experimental procedures.

Echocardiographic Evaluation. In vivo cardiac geometry and function were serially assessed by transthoracic echocardiography (Acuson Sequoia C256, 13 MHz transducer; Siemens) in conscious mice. M-mode LV end-systolic and end-diastolic cross-sectional diameter (LVESD, LVEDD), and the mean of septal and posterior wall thicknesses were determined from an average of 3-5 cardiac cycles. LV fractional shortening (% FS) and LV mass were determined using a cylindrical model as previously described (Barouch et al. (2003)).

Histological Evaluation and Cellular Morphometry. Myocyte cross-sectional diameter was determined from 3-4 different hearts in each group, averaging results from >20 cells per heart. Digitized hematoxylin and eosin stained images were analyzed with Adobe Photoshop 7.0.1. Myocardial fibrosis was determined from Masson trichrome and picrosirius red stained paraffin-embedded myocardial sections, the latter examined using standard as well as polarized light illumination. All slides were scored by a pathologist blinded as to tissue source using a semi-quantitative scale (0=absent; 3=marked fibrosis) (Moens et al. (2008)).

Measurement of NOS Activity. NOS calcium-dependent activity was determined from myocardial homogenates by measuring ¹⁴C arginine to citrulline conversion (assay kits from Stratagene, La Jolla, Calif. or Cayman Chemical, Ann Arbor, Mich.) as previously described (Takimoto et al. (2005)).

Measurement of Total and NOS-Dependent Superoxide Generation. Myocardial superoxide was assayed by lucigenin-enhanced chemiluminescence in snap-frozen LV myocardium. Tissue was homogenized and equilibrated in Krebs-Hepes solution, and after sonification and centrifugation to remove cell debris and nuclei, the supernatant was added to a 5 μM lucigenin-solution, containing 150 μM NADPH. Baseline and maximum lucigenin-enhanced chemiluminescent signal were detected by a liquid scintillation counter (LS6000IC, Beckman Instruments, Fullerton, Calif.), with data reported as counts per minute per milligrams of tissue after background subtraction (cpm/mg) (Moens et al. (2008)). In the same experiment, N (G)-nitro-1-arginine methyl ester (L-NAME, 100 μM) was added to another sample from each heart and the results subtracted from the total to determine NOS-dependent O₂⁻ generation (Kinugawa et al. (2005)).

Western Blot Analysis. Snap frozen heart tissues were homogenized in cell lysis buffer (Cell Signaling Technology, Danvers, Mass.) with 0.01% phosphatase inhibitor cocktails (Sigma, St. Louis, Mo.) and protease inhibitor PMSF (10 mM, Roche, Nutley, N.J.). 60 μg protein was loaded onto 8-16% Tris-Glycine Novex mini-gels (lnvitrogen, Carlsbad, Calif.), electrophoresed and transferred to nitrocellulose or PDVF membranes. 10% SDS/PAGE gels and a semi-dry transfer cell (Bio-Rad, Hercules, Calif.) were used for NOS protein analysis. Primary antibodies were Akt: 1:1000, p-Akt: 1:250 (Cell Signaling, Danvers, Mass.); GTPCH-1:1:500 (a gift from Dr. Shimizu, Showa University, Japan); GAPDH: 1:10,000 (Imgenex, San Diego, Calif.) or 1:500 (Santa Cruz Biotechnology, Santa Cruz, Calif.); eNOS: 1:500 (BD Transduction Laboratories, San Diego, Calif.) or 1:1000 (Santa Cruz Biotechnology, Santa Cruz, Calif.); and p-eNOS (Serine 1177) 1/500 (Cell Signaling Technology, Danvers, Mass.); iNOS: 1:500 (Santa Cruz Biotechnology, Santa Cruz, Calif.); nNOS: 1:500 (Santa Cruz Biotechnology, Santa Cruz, Calif.). Immunoblots were developed on film using enhanced chemiluminescence (SuperSignal West Pico and Femto, Pierce, Rockford, Ill.). Controls included: eNOS+: bovine aortic endothelial cells treated with VEGF; eNOS−: eNOS^{-/-} heart tissue (Jackson Laboratories, Bar Harbor, Me.); nNOS+: rat brain lysate (Santa Cruz Biotechnology, Santa Cruz, Calif.); nNOS−: nNOS^{-/-} heart tissue (Jackson Laboratories, Bar Harbor, Me.); iNOS+: iNOS electrophoresis standard (Cayman Chemical, Ann Arbor, Mich.); and iNOS−: iNOS^{-/-} heart tissue (Jackson Laboratories, Bar Harbor, Me.).

BH4 Measurement. HPLC analysis with fluorescent detection after differential iodine oxidation of tissue extracts in either acidic or alkaline conditions, respectively measured total biopterins (BH4, BH2, and biopterin) and biopterins excluding BH4 (BH2+biopterin). BH4 was calculated as the difference between the two measurements as previously described (Alp et al. (2003)).

Statistical Analysis. Data are expressed as mean±standard error of the mean (SEM). Echocardiographic data were compared using repeated measures analysis of variance (RM-ANOVA), excluding data from the 9 week time point due to survival bias. A Huynh-Feldt correction was chosen since the Mauchly test for sphericity was significant. Kaplan-Meier survival curves were compared using the log rank test. Other data were analyzed using a one-way (or two-way in the case of BH4 treatment group comparisons) ANOVA with a Bonferroni post-hoc test for multiple comparisons, or a Kruskal-Wallis test followed by a Mann-Whitney test for non-parametric data. P-values less than 0.05 were considered to be statistically significant. SPSS version 14.0, Sigmastat 3.0, and GraphPad Prism 5.0 was used for statistical analysis.

Cardioprotective Effect of Beta 3 Adrenoreceptor Agonism in Pressure Overload Induced Hypertrophy—The Role of Neuronal NItrix Oxide Synthase

General Experimental Model. Thirty-eight male C57BL/6J mice (9-10 weeks old, Jackson Laboratory, Bar Harbor, Me.) were randomly divided into 3 groups. Two-thirds of the mice underwent transverse aortic constriction (TAC) to induce cardiac hypertrophy and heart failure via pressure overload as previously described. Takimoto et al. (2005). Briefly, after anesthetized with 2% isoflurane, the chest was opened through a lateral thoracic window between ribs 2 and 4, and a 27 G needle was placed besides the transverse aorta. The band was secured using a 7.0 prolene suture, the needle was then removed and the chest was closed. The remaining third were exposed to sham surgery as control, using the same procedure as TAC without binding the aorta. Half of the TAC mice were treated with BRL (Tocris Bioscience, Ellisville, Mo.) at 0.1 mg/kg/day via osmotic mini-pumps (Alzet Inc, Cupertino, Calif.) which were subcutaneously implanted one day post TAC. The other half of TAC mice received osmotic pump containing only vehicle (PBS). All animals were sacrificed after 3 weeks. Myocardial tissue was either preserved in 10% formalin or snap-frozen in liquid nitrogen for subsequent analysis. Mice were housed in a university animal facility with a 12-hour light-dark cycle and allowed water and food ad libitum. Animal treatment and care was provided in accordance with institutional guidelines. The institutional Animal Care and Use Committee of the Johns Hopkins University School of Medicine approved all protocols and experimental procedures.

Cardiac Function and Geometry. In vivo cardiac geometry and function were serially assessed by transthoracic echocardiography (Acuson Sequoia C256, 13 MHz transducer, Siemens, Oceanside, Calif.) in conscious mice at baseline, 1 week and 3 weeks, respectively. M-mode left ventricular (LV) end-systolic and end-diastolic cross-sectional diameter (LVESD, LVEDD), and the mean of septal and posterior wall thicknesses were determined from an average of 3-5 beats. Left Ventricle (LV) fractional shortening (FS %) and calculated LV mass were determined using a cylindrical model as previously described. Barouch et al. (2003). Echocardiography was evaluated by investigators blinded to the different treatment of groups as described.

Histological Evaluation and Cellular Morphometry. Myocardium was fixed in 10% formalin, processed by routine and standard embedding and serially sectioned in 5-8 um thickness. Myocyte cross-sectional diameter was determined from digitized images of hematoxylin and eosin (H&E) stained slides and analyzed using Image J program (NIH, Besthesda, Md.). Myocardial fibrosis was determined by Masson trichrome staining and was scored by pathologist blinded as to tissue source using a semi-quantitative scale (0=absent; 3=severe fibrosis). Average data reflect results from 3-4 hearts in each group.

Measurement of Cardiac NO Production. Cardiac NO production was determined as the measurement of Nitrate plus Nitrite using Griess reaction assay (assay kit from Cayman Chemical, Ann Arbor, Mich.) as previously described (Saraiva et al. (2005)).

Measurement of Cardiac Superoxide Generation. Myocardial superoxide generation was assayed by lucigenin-enhanced chemiluminescence. Fresh-frozen myocardium was homogenized in 20 mM HEPES buffer containing 1 tablet of mini EDTA-free protease inhibitor cocktail (Roche, Indianapolis, Ind.) and 1 mM PMSF (Roche), then centrifuged at 800 g for 10 minutes at 4° C. to get the supernatant. Supernatants (from at least 4.77 mg tissue) were loaded with Krebs-HEPES buffer (120 mM NaCl, 4.7 mM KCl, 1.2 mM MgSO₄, 1.2 mM KH₂PO₄, 2.5 mM CaCl₂, 25 mM NaHCO₃, and 5.5 mM glucose), 5 uM lucigenin (Sigma Aldrich, St. Louis, Mo.) and 100 uM nicotinamide adenine dinucleotide phosphate-oxidase (NADPH, Sigma Aldrich) to the liquid scintillation counter (LS6000IC, Beckman Instruments, Fullerton, Calif.). Signals were recorded as counts per minute (CPM) and data were normalized to the weight of loaded tissue as CPM/mg tissue. In the same experiment, another part of each tissue was pre-incubated with 100 uM potent nNOS specific inhibitor Vinyl-L-NIO (L-VNIO, Cayman Chemical, Ann Arbor, Mich.) for 30 minutes in cold room to determine the superoxide generation by acute inhibition of nNOS.

Western Blot Analysis. Snap-frozen LV tissue was homogenized in cell lysis buffer (Cell Signaling Technology, Danvers, Mass.) with 0.01% phosphatase inhibitor cocktails (Sigma), 1 tablet of mini EDTA-free protease inhibitor cocktail and protease inhibitor PMSF (1 mM Roche). 60 μg heated protein was separated on 4-12% Bis-Tris NuPAGE Novex mini gel (Invitrogen, Carlsbad, Calif.), electrophoresed and transferred to a PDVF membrane. Phospospecific antibodies eNOS-Ser1177 (1:1000) and -Ser114 (1:1000) were purchased from Cell Signaling Technology (Lake Placid, N.Y.), and p-eNOS Thr495 (1:1000) from BD Biosciences (San Jose, Calif.). eNOS (1:1000), iNOS (1:500), nNOS (1:500), and GAPDH, 1:10000 were purchased from Santa Cruz Biotechnology). The densitometric volume of digitalized band was evaluated by Image J program.

Low-temperature SDS-PAGE was performed to determine eNOS monomer-to-dimer ratio. 50 μg protein with 5-fold Laemmli buffer (0.32 M Tris-HCl, pH 6.8, 0.5 M glycine, 10% SDS, 50% glycerol, and 0.03% bromophenolblue) was loaded onto 7.5% Tric-Glycine ready gel (Bio-Rad) which was running on ice at 100 Volts for 5 hours at 4° C. Then, protein was transferred to PVDF on ice under 14 Volts overnight at 4° C. Subsequent procedures were as same as the regular Western blot.

Statistical Analysis. All data are expressed as mean ±standard error of the mean (SEM). Echocardiographic data were compared using repeated measures analysis of variance (RM-ANOVA). Group data were compared using one-way ANOVA with a Tukey's post-hoc test for multiple comparisons. P values less than 0.05 were considered to be statistically significant. GraphPad Prism 5.0 (La Jolla, Calif.) was used for statistical analysis.

### Example 1

### Baseline Characteristics and Age-Related Hypertrophy in B3^{-/-} Mice

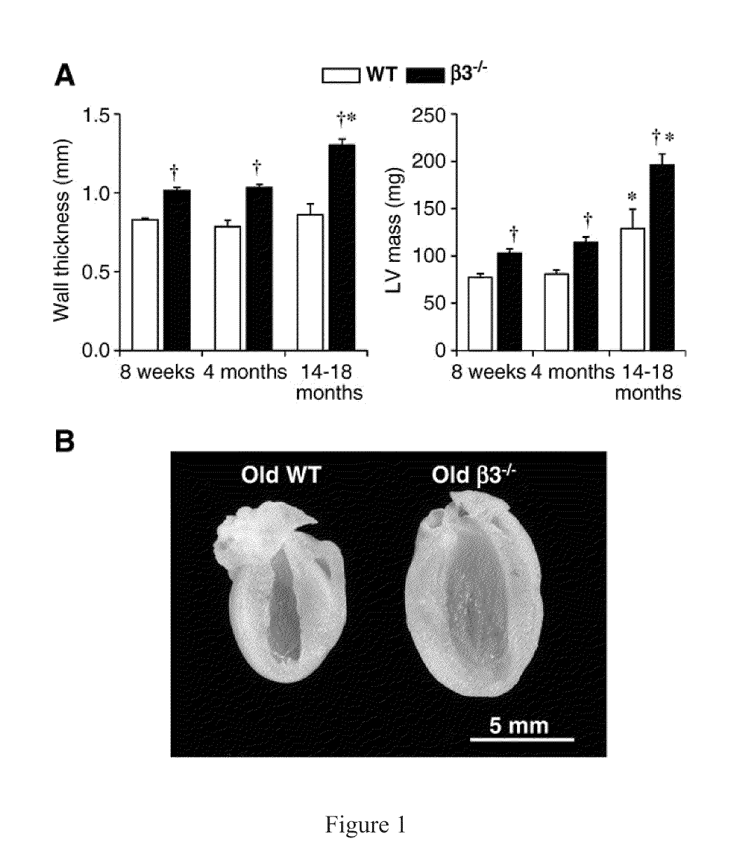

B3^{-/-} mice developed mildly increased body weight, LV wall thickness, and LV mass by echocardiography compared to WT mice by 8 weeks of age. Heart rate, LV dimensions and systolic function are similar between strains (Table 1). The degree of hypertrophy is similar at 8 weeks and 4 months of age (FIG. 1A). In older age (14-18 months old), WT mice develop mild hypertrophy (P<0.05 vs. young WT), however the β3^{-/-} animals have markedly increased LV wall thickness (1.30±0.04 vs. 0.86±0.07 mm, P<0.001) and mass (196±12 vs. 129±20 mg, P<0.05) compared to old WT (FIGS. 1A, B).

TABLE 1
Baseline Characteristics

| | Wildtype | B3^{-/-} |
|---|---|---|
| Body Weight (g) | 2.48 ± 0.3 | 29.3 ± 0.7* |
| Heart Rate (bpm) | 696 ± 16 | 696 ± 10 |
| LV End-Diastolic Diameter (mm) | 2.80 ± 0.06 | 2.81 ± 0.06 |
| LV End-Systolic Diameter (mm) | 1.07 ± 0.02 | 1.12 ± 0.03 |
| Wall Thickness (mm) | 0.94 ± 0.02 | 1.05 ± 0.03* |
| Fractional Shortening (%) | 61.7 ± 0.7 | 60.2 ± 0.9 |
| Calculated LV Mass (mg) | 90 ± 3 | 106 ± 4* |
| LV Systolic Pressure before TAC | 95 ± 5 | 96 ± 4 |
| (mm Hg) | | |
| LV Systolic Pressure after TAC | 137 ± 15 | 130 ± 4 |
| (mm Hg) | | |

LV, left ventricular;
TAC, transverse aortic construction.
*P < 0.05

### Example 2

### Mild Pressure-Overload Increases Mortality, Hypertrophy, and Fibrosis in β3^{-/-} Mice

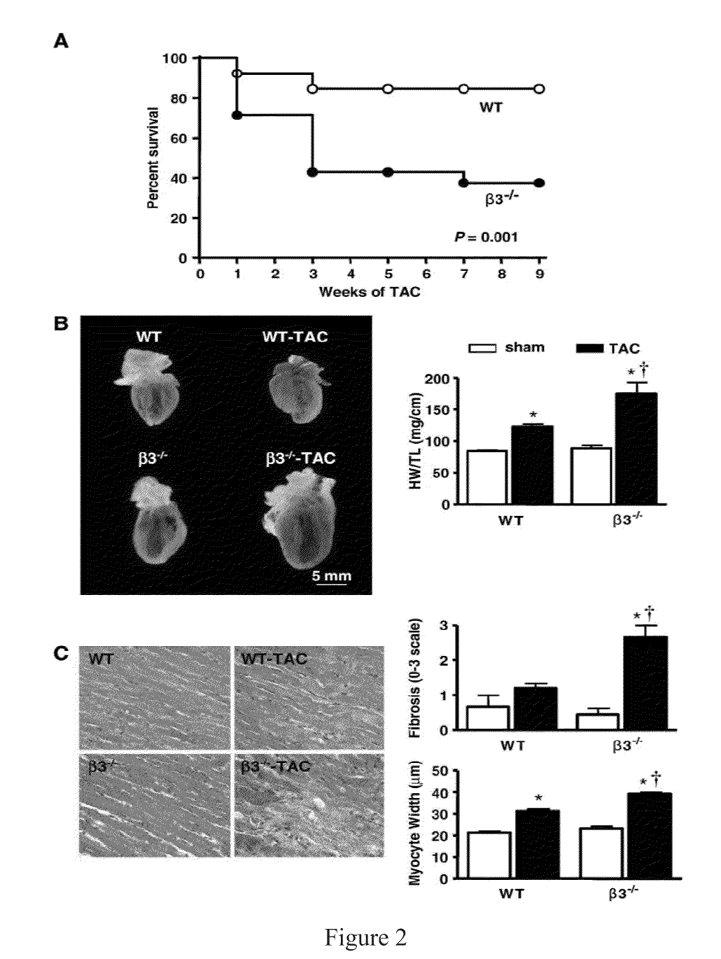

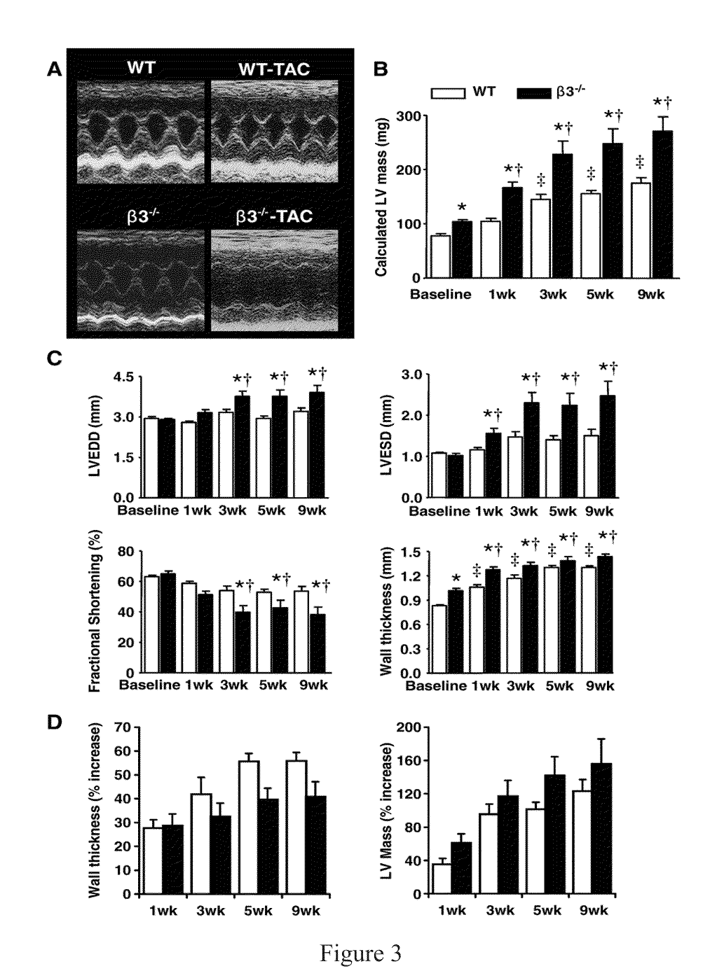

Baseline LV systolic pressures were similar between WT (95±5 mm Hg) and β3^{-/-} (96±4 mm Hg) mice and were increased with mild (25 G) transverse aortic constriction to similar levels (WT-TAC 137±15 mm Hg; β3^{-/-} TAC 130±4 mm Hg) (Table 1). FIG. 2A shows Kaplan-Meier survival curves for both mouse strains following mild TAC. With TAC, 85% of WT animals survived the full 9 week protocol, whereas only 38% of the β3^{-/-} animals did (FIG. 2A, χ²=10.78, P=0.001). The worsened mortality in β3^{-/-} mice was coupled to exacerbated cardiac remodeling, which was mild in WT-TAC versus WT-sham controls (heart weight/tibia length ratio, 123.3±4.0 mg/cm vs. 84.6±2.0 mg/cm, P=0.004), but much greater in β3^{-/-} mice (175.2±17.8 mg/cm vs. 89.0±4.6 mg/cm, P=0.017 vs. β3^{-/-} sham; P=0.003 vs. WT-TAC; FIG. 2B). These findings were paralleled by calculated LV mass based on echocardiography (P=0.001 for WT vs. β3^{-/-} response; FIG. 3B). Although there were baseline differences in calculated LV mass by echocardiography, there was no difference in sham heart weight/tibia length ratio due to the larger size of the β3^{-/-} mice. Myocyte width was significantly greater in β3^{-/-} TAC vs. WT-TAC (39.3±0.9 μm vs. 31.3±0.9 μm, P<0.001), and myocardial fibrosis was also far more pronounced (2.7±0.3 vs. 1.2±0.1, P=0.014; FIG. 2C).

### Example 3

### Pressure-Overload Induces LV Dilation and Dysfunction in β3^{-/-} Mice

β3^{-/-} also developed exacerbated LV chamber dilation and systolic dysfunction, assessed by echocardiography (FIG. 3A) in response to pressure-overload. After 9 weeks of TAC, LVEDD was unchanged in WT mice but increased in β3^{-/-} mice vs. baseline (3.90±0.26 vs. 2.91±0.04 mm, P=0.001). Similarly, LVESD was increased vs. baseline (2.47±0.36 mm vs. 1.02±0.05 mm P_{interaction}<0.001), with a net decline in fractional shortening (38.2±5.0 vs. 64.9±1.8%, P=0.002) in β3^{-/-} TAC but not WT-TAC. Average wall thickness increased in both WT-TAC (1.30±0.02 vs. 0.83±0.01 mm, P<0.001) and β3^{-/-} TAC mice, but was higher in β3^{-/-} TAC (1.43±0.03 vs. 1.02±0.03 mm, P<0.001 vs. β3^{-/-} sham, P<0.01 vs. WT-TAC; FIG. 3C), although percent increase in wall thickness was similar between strains due to the baseline hypertrophy in the β3^{-/-} mice. Likewise, percent increase in LV mass was similar in β3^{-/-} and WT (FIG. 3D).

### Example 4

### Lack of β3-AR Alters NOS Isoform Expression and Decreases NOS Activity with TAC

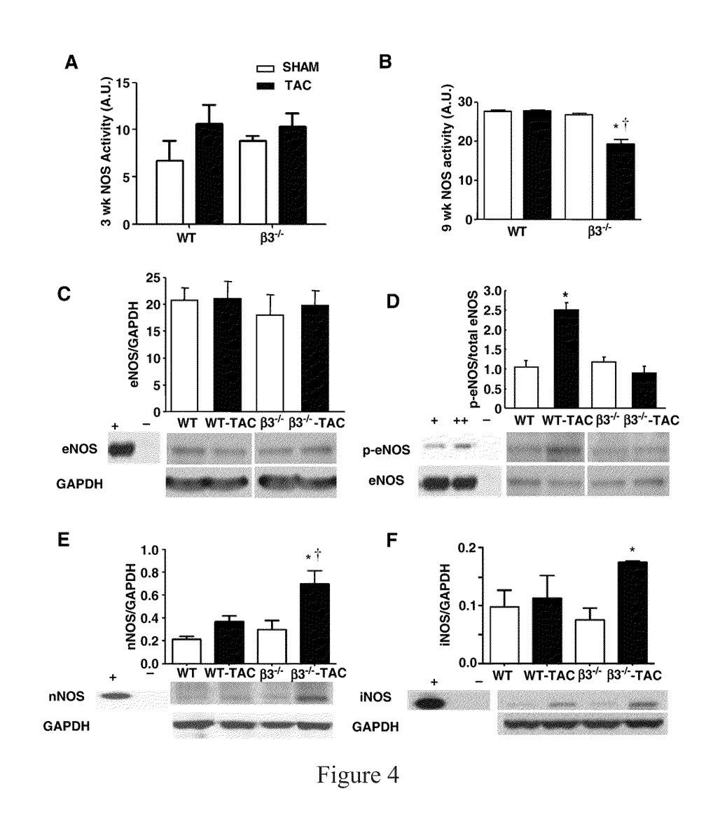

Since β3-AR cardiac modulation is coupled to NOS, whether mice lacking the receptor had decreased NOS activity was examined. After 3 weeks of TAC, there were no significant differences in arginine-citrulline conversion (FIG. 4A) from baseline in either WT-TAC (10.6±2.0 vs. 6.7±2.1; arbitrary units (A.U.), P=NS) or β3^{-/-} TAC (10.3±1.4 vs. 8.8±0.6; A.U., P=NS). At 9 weeks, NOS activity was similar between β3^{-/-} and WT sham (26.9±0.4 vs. 27.6±0.4; A.U., P=NS). Mild pressure-overload did not alter NOS activity in WT-TAC (27.7±0.3; A.U.) but it decreased activity in β3^{-/-} TAC (19.3±1.2; A.U., P<0.001; FIG. 4B) after 9 weeks. This decline was not associated with reduced eNOS protein expression (FIG. 4C). However, S1177 phosphorylation, an indication of eNOS activation, was increased in WT-TAC. In contrast, β3^{-/-} showed no increase in p-eNOS with TAC (FIG. 4D). Furthermore, an increase in total nNOS expression in the β3^{-/-} TAC hearts was noticed compared to baseline levels (P<0.05, FIG. 4E) and levels seen in WT (P<0.05). iNOS protein levels also increased in β3^{-/-} TAC above baseline (P<0.01, FIG. 4F), though this was not significantly different from levels in WT controls.

### Example 5

### Lack of β3-AR Results in Greater NOS Uncoupling with TAC

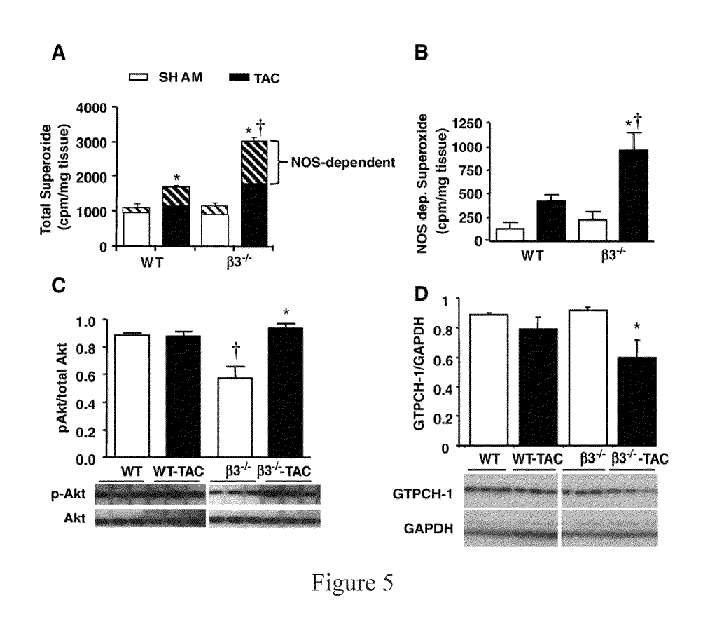

Reduced NOS activity can also be due to its functional uncoupling, wherein the enzyme shifts to generate superoxide rather than NO. To test for this, lucigenin-enhanced chemiluminescence in myocardium was examined in the presence and absence of the NOS inhibitor L-NAME. Superoxide was similar in both genotypes 9 weeks following sham surgery (1145±146 vs. 1106±109 cpm/mg, P=NS) but rose almost twice as much in β3^{-/-} compared to WT mice after 9 weeks of TAC (2730±121 vs. 1719±52 cpm/mg; P<0.05 vs. baseline, P<0.001 between groups; FIG. 5A). Importantly, the dominant component of enhanced O₂⁻ in β3^{-/-} TAC could be attributed to NOS uncoupling, although both NOS-dependent and NOS-independent superoxide were increased in β3^{-/-} TAC hearts. NOS-dependent superoxide was similar between β3^{-/-} and WT at baseline, although there was a trend toward higher levels in the β3^{-/-}; however, levels rose nearly 300% in β3^{-/-} TAC mice vs. β3^{-/-} at baseline, compared with <200% in WT-TAC vs. WT at baseline; P<0.05 for both (FIG. 5B). In addition, NOS-dependent superoxide was higher in β3^{-/-} TAC vs. WT-TAC (P<0.01).

Because Akt can modulate eNOS phosphorylation, whether it was differentially phosphorylated (S476) was examined. Although basal Akt phosphorylation was reduced in β3^{-/-} mice, it rose with 9 weeks of TAC to similar levels in both genotypes (FIG. 5C), indicating that p-eNOS and NOS activity must be regulated by a non-Akt dependent mechanism.

NOS coupling depends directly upon levels of tetrahydrobiopterin (BH4), whose rate-limiting synthetic enzyme is guanosine triphosphate cyclohydrolase 1 (GTPCH-1). Whether GTPCH-1 expression was altered in the β3^{-/-} model was therefore tested. GTPCH-1 expression was similar at baseline but declined significantly after 9 weeks of TAC in β3^{-/-} TACvs. β3^{-/-} sham (P<0.05; FIG. 5D).

### Example 6

### Tetrahydrobiopterin (BH4) Treatment Rescues β3^{-/-} Mice from LV Hypertrophy and Dysfunction Following Pressure-Overload

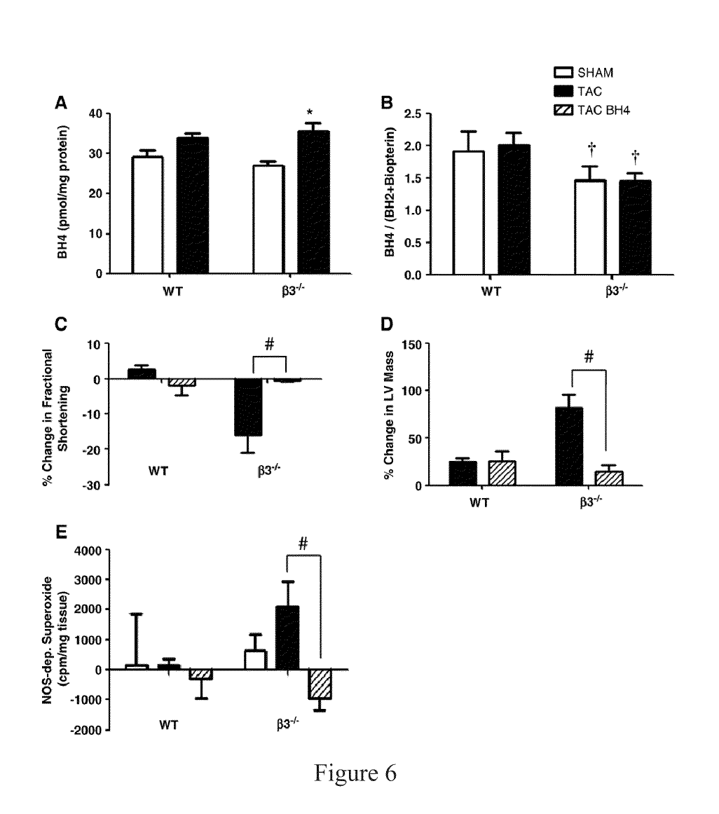

Given the decrease in GTPCH-1 protein levels, whether BH4 levels might differ in the β3^{-/-} mice, either at baseline or in response to TAC, was considered. Using HPLC to fraction biopterins, total BH4 levels did not differ significantly between strains, although there was a slight increase (P<0.01, FIG. 6A) in β3^{-/-} TAC (35.6±1.9 pmol/mg protein) above baseline (27.0±0.9 pmol/mg protein). The ratio of BH4 to other biopterins (BH2+biopterin) was decreased by approximately 25% (P=0.03) in β3^{-/-} mice at baseline (1.49±0.2) compared to WT (1.91±0.3), yet was unchanged after TAC (FIG. 6B).

Given the greater amounts of NOS-dependent O₂⁻ generated in β3^{-/-} TAC, whether exogenously adding BH4 might be a viable therapeutic strategy, as has been reported previously in systems of uncoupled NOS (Moen et al. (2008)), was tested. BH4 or vehicle was therefore supplemented to the feed of β3^{-/-} and WT mice and TAC or sham surgery was performed. This cohort of mice was sacrificed after 3 weeks of TAC, in order to minimize any survival bias or secondary pathway activation that might be more significant at later time points. After 3 weeks of TAC, β3^{-/-} TAC mice experienced a decrease in fractional shortening (−16.1±4.9%, FIG. 6C) and increase in LV mass (+81.8±13.7%, FIG. 6D) as estimated by echocardiography. BH4 treatment completely rescued the impairment in function, with no change in fractional shortening in β3^{-/-} TAC/BH4 (−0.4±0.2%, P<0.05), similar to WT-TAC (+2.5±1.2%) and WT-TAC/BH4 (−1.8±3.0%) controls (P=NS for both). Similarly, the increase in calculated LV mass was much lower in β3^{-/-} TAC/BH4 (+15.0±6.8%, P<0.01 vs. β3^{-/-} TAC) to a level not significantly different from WT (FIG. 6D).

Recognizing the dramatic protection of BH4 treatment from pathological hypertrophy and impaired systolic function induced by TAC, it was hypothesized that this protection might correlate with a decrease in NOS-dependent superoxide production. Indeed, after 3 weeks of TAC, BH4 treatment reduced NOS-dependent superoxide production in whole heart homogenates (P<0.05) to a level similar to baseline and WT-TAC controls (FIG. 6E).

### Example 7

### β3 Adrenocreceptor Deterioration of Cardiac Function

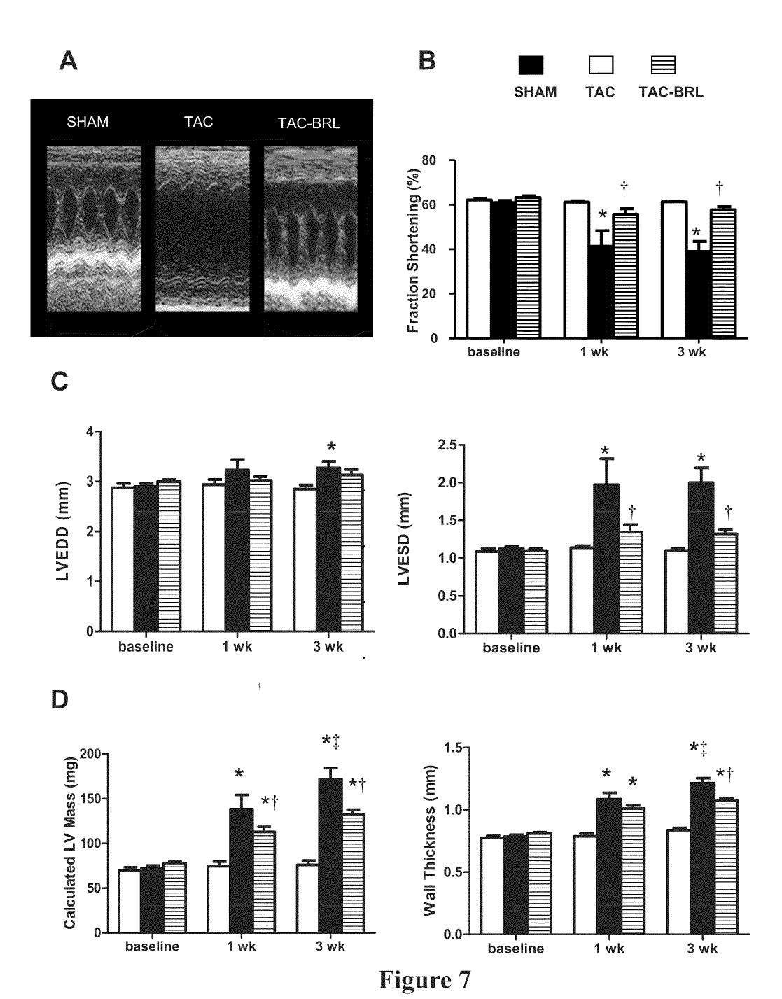

Mice developed increased LV chamber dilation and systolic dysfunction after 3 weeks of TAC (FIG. 7A), as evidenced by 82% increased LVESD (2.00±0.20 vs. 1.10±0.03 mm; P<0.001) and 36% reduction in FS % (39.1±4.5 vs. 61.4±0.3%; P<0.001) compared to sham mice by echocardiography (FIGS. 7B, C). Calculated LV mass (172±13 vs. 76±5 mg; P<0.001) and average wall thickness (1.21±0.04 vs. 0.84±0.02 mm; P<0.001) were increased vs. sham (FIG. 7D). Three weeks of BRL treatment via subcutaneous osmotic pumps at 0.1 mg/kg/day totally prevented LV dilation (LVESD 1.32±0.06; P=NS vs. sham, P<0.01 vs. TAC), and cardiac systolic function remained normal (FS % 57.8±1.4; P=NS vs. sham, P<0.001 vs. TAC). Calculated LV mass and average wall thickness were significantly lower in BRL treated mice compared to vehicle (P<0.001 vs. TAC).

### Example 8

### β3 Adrenoreceptor Reduced Development of Cardiac Hypertrophy

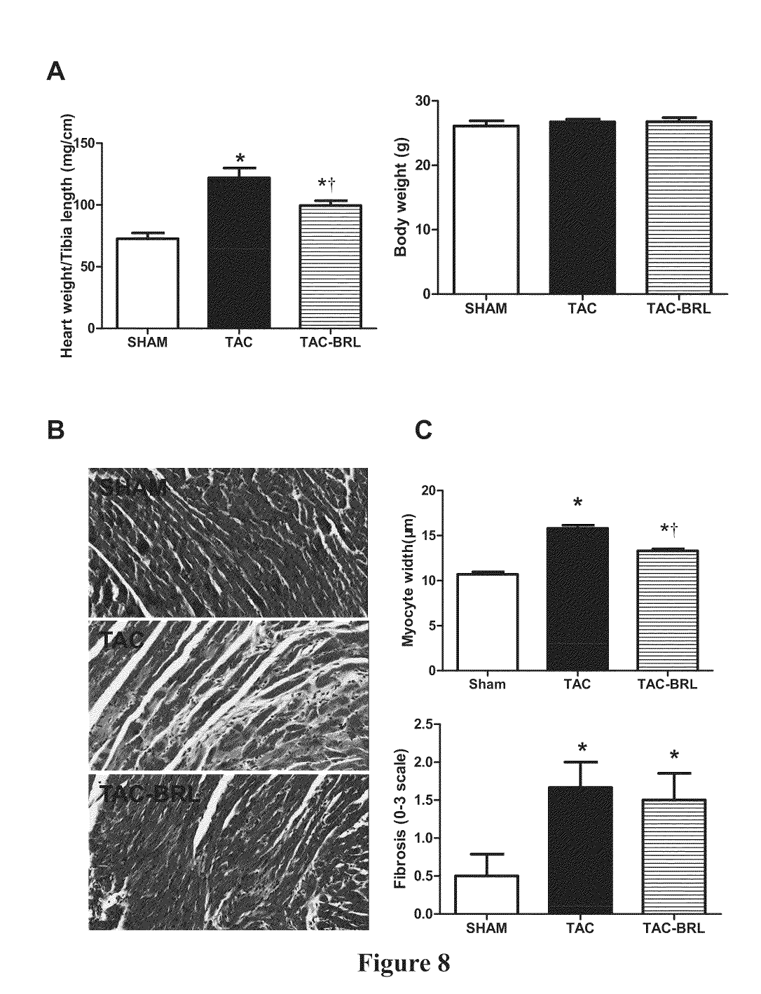

Three weeks of TAC resulted in increased cardiac hypertrophy vs. sham, with 67% higher heart weight to tibia length ratio (HW/TL 122±8 vs. 73±5 mg/cm; P<0.001). BRL treated mice developed less hypertrophy (HW/TL 100±4 mg/cm; P<0.05 vs. vehicle (FIG. 8A). These findings were paralleled by similar changes in calculated LV mass by echocardiography (FIG. 7D). Both cardiomyocyte width by H&E staining (15.81±0.35 vs. 10.71±0.26 μm; P<0.001) and fibrosis scale (0-3 scale; 0=none, 3=severe) by Trichrome staining (1.67±0.33 vs. 0.50±0.29; P<0.05) were significantly greater in TAC vs. sham. Interestingly, BRL reduced cardiomyocyte width (13.31±0.21 μm; P<0.001 vs. TAC) but had no effect on fibrosis scale (1.50±0.35; P=NS vs. TAC; FIGS. 8B, C).

### Example 9

### β3 Adrenoreceptor Stimulation Increases Cardiac NO Production and Decreases ROS Production in Pressure-Overloaded Mice

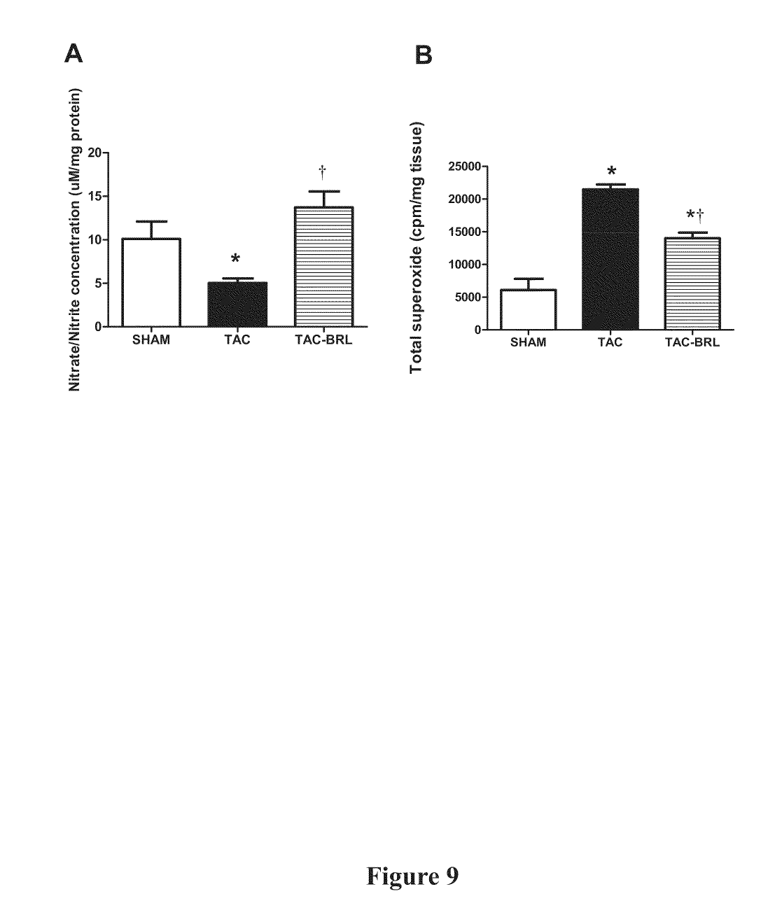

β3-AR induced negative inotropic effect was thought to be associated with NO release via NOS (Gauthier et al. (1998)). Previous data showed that mice lacking β3-AR had lower NOS activity and generated more cardiac superoxide than WT mice after pressure-overload (Moens et al. (2009)). NO production was therefore tested by measuring total nitrate/nitrite concentration by using Griess assay and the superoxide generation by lucigenin-enhanced chemiluminescence assay to observe β3-AR agonism on NO and ROS production. The total nitrate/nitrite concentration was decreased 50% (5.03±0.52 vs. 10.10±1.99 μM/mg protein; P<0.05; FIG. 9A) and superoxide was increased ˜3.5 fold (21459±783 vs. 6099±1703 CPM/mg tissue; P<0.001; FIG. 9B) in TAC hearts over sham controls. Three weeks of BRL treatment restored nitrate/nitrite concentration back to normal (13.73±1.84 μM/mg protein) and partially inhibited superoxide generation (14017±838; P<0.01 vs TAC for both).

### Example 10

### in Adrenoreceptor Stimulation Increases nNOS Protein Expression

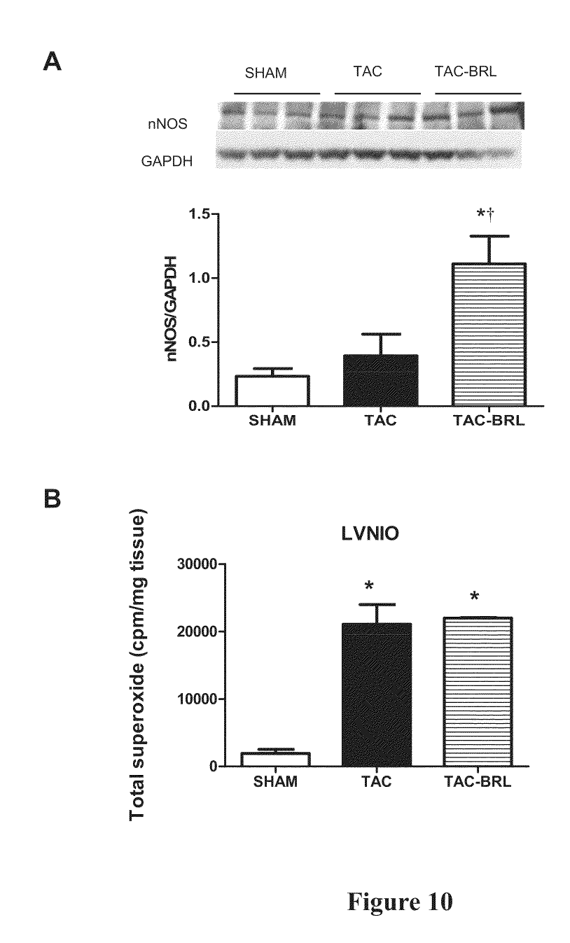

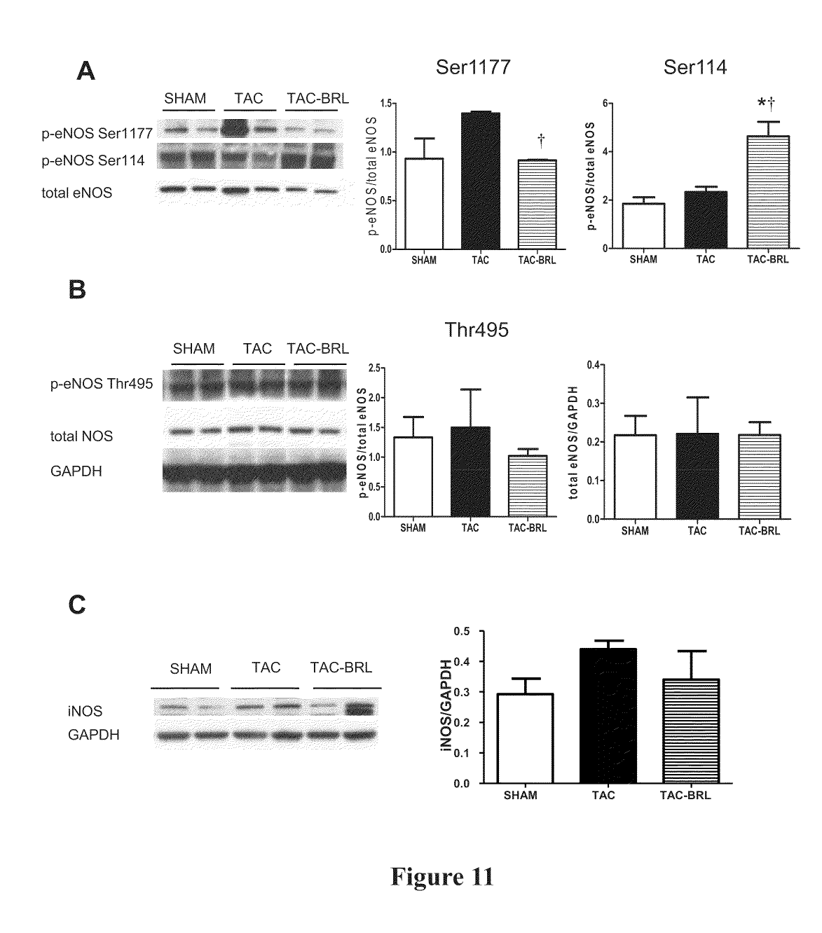

Recent experiments demonstrated that nNOS derived NO production was involved in altered contractile response by β3-AR stimulation in both diabetic and senescent heart (Birenbaum et al. (2008); Amour et al. (2007)). An almost 2-fold increase of nNOS protein expression in BRL treated compared to vehicle heart was observed (1.11±0.22 vs. 0.39±0.17 arbitrary units (A.U.); P<0.05; FIG. 10A), though there was no difference between sham and TAC. More interestingly, when pretreated LV homogenate with 100 nM specific nNOS inhibitor L-VNIO, the suppression of superoxide generation by BRL was abolished (21992±76 vs. 21063±2930 CPM/mg tissue; P=NS vs. TAC; FIG. 10B).

### Example 11

### BRL Modulation of eNOS Activation

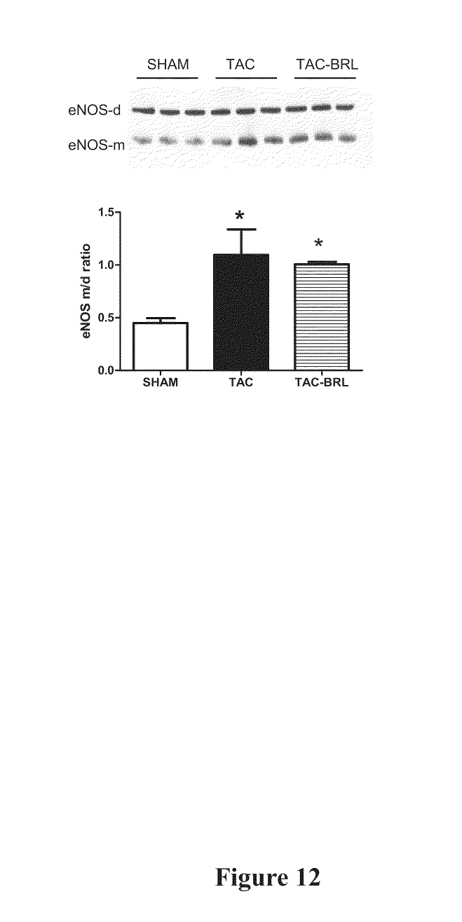

To further investigate the role of BRL on other NOS isoforms, eNOS protein expression and phosphorylation was examined. There are three enzyme phosphorylation sites that have been shown to modulate eNOS activity: eNOS^{Ser1177}, eNOS^{Ser114} and eNOS^{Thr495}. After 3 weeks of TAC+BRL treatment, eNOS^{Ser1177} phosphorylation, which indicates eNOS activation, was decreased compared to TAC alone (0.92±0.01 vs. 1.40±0.02 A.U.; P<0.01), though there was no change between sham and TAC. In contrast, phosphorylation of eNOS^{Ser114} which is an indication of eNOS deactivation, was increased 100% in BRL treated mice (P<0.05 vs. TAC), though levels were similar between sham and TAC (FIG. 11A). Both eNOS^{Thr495} phosphorylation and total eNOS protein expression were unchanged between groups (FIG. 11B). eNOS^{Ser635} were similar between groups as well (data not shown). There was a trend toward up-regulation of inducible NOS (iNOS) protein level after 3 weeks of TAC (0.44±0.03 vs. 0.29±0.05 A.U.; P=0.06 vs. sham); however, BRL had no effect on iNOS expression (0.34±0.09 A.U.; P=NS vs. TAC; FIG. 11C).

### Example 12

### BRL Modulation on eNOS Dimerization

eNOS homodimer coupling condition indexed by eNOS monomer to dimer ratio (m/d) is an indication for eNOS uncoupling. Uncoupled eNOS switches NO generation to superoxide generation. Three weeks of TAC resulted in increased eNOS uncoupling (m/d 1.10±0.24 vs. 0.45±0.05 A.U.; P<0.05) which is consistent with previous reports (Moens et al. (2008); Moens et al. (2006); Takimoto et al. (2005)). Three weeks of BRL treatment did not change the m/d ratio (1.01±0.02 A.U.; P=NS vs. TAC; FIG. 12).

### References

N. J. Alp, S. Mussa, J. Khoo, S. Cai, T. Guzik and A. Jefferson et al., Tetrahydrobiopterin-dependent preservation of nitric oxide-mediated endothelial function in diabetes by targeted transgenic GTP-cyclohydrolase I overexpression, J. Clin. Invest. 112 (2003), pp. 725-735.

J. Amour, X. Loyer, M. Le Guen, N. Mabrouk, J. S. David and E. Camors et al., Altered contractile response due to increased beta3-adrenoceptor stimulation in diabetic cardiomyopathy: the role of nitric oxide synthase 1-derived nitric oxide, Anesthesiology 107 (2007), pp. 452-460.

Arch J R. The discovery of drugs for obesity, the metabolic effects of leptin and variable receptor pharmacology: perspectives from beta3-adrenoceptor agonists. Naunyn Schmiedebergs Arch Pharmacol. August 2008; 378(2):225-240.

Arch J R. beta(3)-Adrenoceptor agonists: potential, pitfalls and progress. Eur. J. Pharmacol. 2002; 440: 99-107.

Bajcetic M, Kokic Nikolic A, Djukic M, et al. Effects of carvedilol on left ventricular function and oxidative stress in infants and children with idiopathic dilated cardiomyopathy: a 12-month, two-center, open-label study. Clin Ther. April 2008; 30(4):702-714.

L. A. Barouch, D. E. Berkowitz, R. W. Harrison, C. P. O'Donnell and J. M. Hare, Disruption of leptin signaling contributes to cardiac hypertrophy independently of body weight in mice, Circulation 108 (2003), pp. 754-759.

L. A. Barouch, R. W. Harrison, M. W. Skaf, G. O. Rosas, T. P. Cappola and Z. A. Kobeissi et al., Nitric oxide regulates the heart by spatial confinement of nitric oxide synthase isoforms, Nature 416 (2002), pp. 337-339.

C. Belge, B. Sekkali, G. Tavernier, A. C. Poulier, L. Bertrand and J. L. Vanoverschelde et al., Cardiomyocyte-specific overexpression of beta3-adrenoceptors attenuates the hypertrophic response to catecholamines in vivo (abstract), Circulation 116 (2007), p. 11_{—}148.

Bendall J K, Damy T, Ratajczak P, et al. Role of myocardial neuronal nitric oxide synthase-derived nitric oxide in beta-adrenergic hyporesponsiveness after myocardial infarction-induced heart failure in rat. Circulation. 2004; 110:2368-2375.

Birenbaum A., Tesse A., Loyer X., et al. Involvement of beta 3-adrenoceptor in altered beta-adrenergic response in senescent heart: role of nitric oxide synthase 1-derived nitric oxide. Anesthesiology. 2008; 109:1045-1053.

Y. C. Boo, H. J. Kim, H. Song, D. Fulton, W. Sessa and H. Jo, Coordinated regulation of endothelial nitric oxide synthase activity by phosphorylation and subcellular localization, Free Radic. Biol. Med. 41 (2006), pp. 144-153.

M. R. Bristow, R. Ginsburg, W. Minobe, et al. Decreased catecholamine sensitivity and beta-adrenergic-receptor density in failing human hearts. N Engl J Med. Jul. 22 1982; 307(4):205-211.

K. Brixius, W. Bloch, C. Ziskoven, B. Bolck, A. Napp and C. Pott et al., Beta3-adrenergic eNOS stimulation in left ventricular murine myocardium, Can. J. Physiol. Pharmacol. 84 (2006), pp. 1051-1060.

K. Brixius, W. Bloch, C. Pott, A. Napp, A. Krahwinkel and C. Ziskoven et al., Mechanisms of beta 3-adrenoceptor-induced eNOS activation in right atrial and left ventricular human myocardium, Br. J. Pharmacol. 143 (2004), pp. 1014-1022.

T. Damy, P. Ratajczak, A. M. Shah, E. Camors, I. Marty and G. Hasenfuss et al., increased neuronal nitric oxide synthase-derived NO production in the failing human heart, Lancet 363 (2004), pp. 1365-1367.

T. Damy, P. Ratajczak, E. Robidel, J. K. Bendall, P. Oliviero and J. Boczkowski et al., Up-regulation of cardiac nitric oxide synthase 1-derived nitric oxide after myocardial infarction in senescent rats, FASEB J. 17 (2003), pp. 1934-1936.

Dawson D, Lygate C A, Zhang M H, et al. nNOS gene deletion exacerbates pathological left ventricular remodeling and functional deterioration after myocardial infarction. Circulation. 2005; 112:3729-3737.

L. J. Emorine, S. Marullo, M. M. Briend-Sutren, G. Patey, K. Tate and C. Delavier-Klutchko et al., Molecular characterization of the human beta 3-adrenergic receptor, Science 245 (1989), pp. 1118-1121.

R. T. Gan, W. M. Li, C. H. Xiu, J. X. Shen, X. Wang and S. Wu et al., Chronic blocking of beta 3-adrenoceptor ameliorates cardiac function in rat model of heart failure, Chin. Med. J. (Engl.) 120 (2007), pp. 2250-2255.

C. Gauthier, C. Seze-Goismier and B. Rozec, Beta 3-adrenoceptors in the cardiovascular system, Clin. Hemorheol. Microcirc. 37 (2007), pp. 193-204.

C. Gauthier, V. Leblais, L. Kobzik, J. N. Trochu, N. Khandoudi and A. Bril et al., The negative inotropic effect of beta3-adrenoceptor stimulation is mediated by activation of a nitric oxide synthase pathway in human ventricle, J. Clin. Invest. 102 (1998), pp. 1377-1384.

C. Gauthier, G. Tavernier, F. Charpentier, D. Langin and H. Le Marec, Functional beta3-adrenoceptor in the human heart, J. Clin. Invest. 98 (1996), pp. 556-562.

R. Germack and J. M. Dickenson, Induction of beta3-adrenergic receptor functional expression following chronic stimulation with noradrenaline in neonatal rat cardiomyocytes, J. Pharmacol. Exp. Ther. 316 (2006), pp. 392-402.

G. A. Haywood, P. S. Tsao, H. E. von der Leyen, M. J. Mann, P. J. Keeling and P. T. Trindade et al., Expression of inducible nitric oxide synthase in human heart failure, Circulation 93 (1996), pp. 1087-1094.

D. S. Hutchinson, E. Chernogubova, O. S. Dallner, B. Cannon and T. Bengtsson, Beta-adrenoceptors, but not alpha-adrenoceptors, stimulate AMP-activated protein kinase in brown adipocytes independently of uncoupling protein-1, Diabetologia 48 (2005), pp. 2386-2395.

F. Ichinose, K. D. Bloch, J. C. Wu, R. Hataishi, H. T. Aretz and M. H. Picard et al., Pressure overload-induced LV hypertrophy and dysfunction in mice are exacerbated by congenital NOS3 deficiency, Am. J. Physiol. Heart Circ. Physiol. 286 (2004), pp. H1070-H1075.

W. Idigo, M. H. Zhang, Y. H. Zhang and B. Casadei, The negative inotropic effect of beta(3)-adrenergic receptor stimulation in nNOS(−/−) mice is restored by oxypurinol (abstract), Heart 92 (2006), pp. A88-A89.

S. Imbrogno, T. Angelone, C. Adamo, E. Pulera, B. Tota and M. C. Cerra, Beta3-adrenoceptor in the eel (Anguilla anguilla) heart: negative inotropy and NO-cGMP-dependent mechanism, J. Exp. Biol. 209 (2006), pp. 4966-4973.

Kass D A. Rescuing a failing heart: putting on the squeeze. Nat Med. January 2009; 15(1):24-25.

Khan S A, Lee K, Minhas K M, et al. Neuronal nitric oxide synthase negatively regulates xanthine oxidoreductase inhibition of cardiac excitation-contraction coupling. Proc. Natl. Acad. Sci. U.S.A. 2004; 101:15944-15948.

Kawai K, Qin F, Shite J, et al. Importance of antioxidant and antiapoptotic effects of beta-receptor blockers in heart failure therapy. Am J Physiol Heart Circ Physiol. September 2004; 287(3):H1003-1012.

S. Kinugawa, H. Huang, Z. Wang, P. M. Kaminski, M. S. Wolin and T. H. Hintze, A defect of neuronal nitric oxide synthase increases xanthine oxidase-derived superoxide anion and attenuates the control of myocardial oxygen consumption by nitric oxide derived from endothelial nitric oxide synthase, Circ. Res. 96 (2005), pp. 355-362.

T. Kitamura, K. Onishi, K. Dohi, T. Okinaka, N. Isaka and T. Nakano, The negative inotropic effect of beta3-adrenoceptor stimulation in the beating guinea pig heart, J. Cardiovasc. Pharmacol. 35 (2000), pp. 786-790.

J. Klein, M. Fasshauer, M. Ito, B. B. Lowell, M. Benito and C. R. Kahn, beta(3)-adrenergic stimulation differentially inhibits insulin signaling and decreases insulin-induced glucose uptake in brown adipocytes, J. Biol. Chem. 274 (1999), pp. 34795-34802.

Liao Y, Asakura M, Takashima S, et al. Celiprolol, a vasodilatory beta-blocker, inhibits pressure overload-induced cardiac hypertrophy and prevents the transition to heart failure via nitric oxide-dependent mechanisms in mice. Circulation. Aug. 10 2004; 110(6):692-699.

A. Maffei, A. Di Pardo, R. Carangi, et al. Nebivolol induces nitric oxide release in the heart through inducible nitric oxide synthase activation. Hypertension. 2007; 50:652-656.

Mallat Z, Philip I, Lebret M, et al. Elevated levels of 8-iso-prostaglandin F2alpha in pericardial fluid of patients with heart failure: a potential role for in vivo oxidant stress in ventricular dilatation and progression to heart failure. Circulation. Apr. 28 1998; 97(16):1536-1539.

P. B. Massion, O. Feron, C. Dessy and J. L. Balligand, Nitric oxide and cardiac function: ten years after, and continuing, Circ. Res. 93 (2003), pp. 388-398.

A L Moens, Leyton-Mange J S, Niu X, et al. Adverse ventricular remodeling and exacerbated NOS uncoupling from pressure-overload in mice lacking the beta3-adrenoreceptor. J Mol Cell Cardiol. November 2009; 47(5):576-585.

A. L. Moens, H. C. Champion, M. J. Claeys, B. Tavazzi, P. M. Kaminski and M. S. Wolin et al., High-dose folic acid pretreatment blunts cardiac dysfunction during ischemia coupled to maintenance of high-energy phosphates and reduces postreperfusion injury, Circulation 117 (2008), pp. 1810-1819.

A. L. Moens, E. Takimoto, C. G. Tocchetti, K. Chakir, D. Bedja and G. Cormaci et al., Reversal of cardiac hypertrophy and fibrosis from pressure overload by tetrahydrobiopterin: efficacy of recoupling nitric oxide synthase as a therapeutic strategy, Circulation 117 (2008), pp. 2626-2636.

A. L. Moens and D. A. Kass, Tetrahydrobiopterin and cardiovascular disease, Arterioscler. Thromb. Vasc. Biol. 26 (2006), pp. 2439-2444.

M. Mongillo, C. G. Tocchetti, A. Terrin, V. Lissandron, Y. F. Cheung and W. R. Dostmann et al., Compartmentalized phosphodiesterase-2 activity blunts beta-adrenergic cardiac inotropy via an NO/cGMP-dependent pathway, Circ. Res. 98 (2006), pp. 226-234.

S. Moniotte, C. Beige, B. Sekkali, P. B. Massion, B. Rozec and C. Dessy et al., Sepsis is associated with an upregulation of functional beta3 adrenoceptors in the myocardium, Eur. J. Heart Fail. 9 (2007), pp. 1163-1171.

S. Moniotte and J. L. Balligand, Potential use of beta(3)-adrenoceptor antagonists in heart failure therapy, Cardiovasc. Drug Rev. 20 (2002), pp. 19-26.

S. Moniotte, L. Kobzik, O. Feron, J. N. Trochu, C. Gauthier and J. L. Balligand, Upregulation of beta(3)-adrenoceptors and altered contractile response to inotropic amines in human failing myocardium, Circulation 103 (2001), pp. 1649-1655.

A. Morimoto, H. Hasegawa, H. J. Cheng, W. C. Little and C. P. Cheng, Endogenous beta3-adrenoreceptor activation contributes to left ventricular and cardiomyocyte dysfunction in heart failure, Am. J. Physiol. Heart Circ. Physiol. 286 (2004), pp. H2425-H2433.

C. Nahmias, N. Blin, J. M. Elalouf et al., Molecular characterization of the mouse beta 3-adrenergic receptor: relationship with the atypical receptor of adipocytes. EMBO J. 1991; 10:3721-3727.

F. Nantel, H. Bonin, L. J. Emorine, V. Zilberfarb, A. D. Strosberg and M. Bouvier et al., The human beta 3-adrenergic receptor is resistant to short term agonist-promoted desensitization, Mol. Pharmacol 43 (1993), pp. 548-555.

A. Napp, K. Brixius, C. Pott, et al. Effects of the beta3-adrenergic agonist BRL 37344 on endothelial nitric oxide synthase phosphorylation and force of contraction in human failing myocardium. J. Card. Fail. 2009; 15:57-67.

C. Pott, K. Brixius, W. Bloch, C. Ziskoven, A. Napp and R. H. Schwinger, Beta3-adrenergic stimulation in the human heart: signal transduction, functional implications and therapeutic perspectives, Pharmazie 61 (2006), pp. 255-260.

Rasmussen H H, Figtree G A, Krum H, et al. The use of beta3-adrenergic receptor agonists in the treatment of heart failure. Curr Opin Investig Drugs. September 2009; 10(9):955-962.

B. Rozec, M. Erfanian, K. Laurent, et al. Nebivolol, a vasodilating selective beta(1)-blocker, is a beta(3)-adrenoceptor agonist in the nonfailing transplanted human heart. J Am Coll Cardiol. Apr. 28 2009; 53(17):1532-1538.

B. Rozec, J. Noireaud, J. N. Trochu and C. Gauthier, Place of beta 3-adrenoceptors among other beta-adrenoceptor subtypes in the regulation of the cardiovascular system, Arch. Mal. Coeur Vaiss. 96 (2003), pp. 905-913.

Saraiva R M, Minhas K M, Raju S V, et al., Deficiency of neuronal nitric oxide synthase increases mortality and cardiac remodeling after myocardial infarction: role of nitroso-redox equilibrium. Circulation. 2005; 112:3415-3422.

Sawyer D B, Siwik D A, Xiao L, et al. Role of oxidative stress in myocardial hypertrophy and failure. J Mol Cell Cardiol. April 2002; 34(4):379-388.

Sharma V, Parsons H, Allard M F, et al. Metoprolol increases the expression of beta(3)-adrenoceptors in the diabetic heart: effects on nitric oxide signaling and forkhead transcription factor-3. Eur J Pharmacol. Oct. 24 2008; 595(1-3):44-51.

Sheeran F L, Rydstrom J, Shakhparonov M I, et al. Diminished NADPH transhydrogenase activity and mitochondrial redox regulation in human failing myocardium. Biochim Biophys Acta. June-July 2010; 1797(6-7): 1138-1148.

P. M. Simard, C. Atgie, P. Mauriege, F. D'Allaire and L. J. Bukowiecki, Comparison of the lipolytic effects of norepinephrine and BRL 37344 in rat brown and white adipocytes, Obes. Res. 2 (1994), pp. 424-431.

S. Takayama, Y. Furukawa, L. M. Ren, Y. Inoue, S. Sawaki and S. Chiba, Positive chronotropic and inotropic responses to BRL 37344, a beta 3-adrenoceptor agonist in isolated, blood-perfused dog atria, Eur. J. Pharmacol. 231 (1993), pp. 315-321.

E. Takimoto and D. A. Kass, Role of oxidative stress in cardiac hypertrophy and remodeling, Hypertension 49 (2007), pp. 241-248.

E. Takimoto, H. C. Champion, M. Li, S. Ren, E. R. Rodriguez and B. Tavazzi et al., Oxidant stress from nitric oxide synthase-3 uncoupling stimulates cardiac pathologic remodeling from chronic pressure load, J. Clin. Invest. 115 (2005), pp. 1221-1231.

G. Tavernier, G. Toumaniantz, M. Erfanian, M. F. Heymann, K. Laurent and D. Langin et al., beta3-Adrenergic stimulation produces a decrease of cardiac contractility ex vivo in mice overexpressing the human beta3-adrenergic receptor, Cardiovasc. Res. 59 (2003), pp. 288-296.

P. Varghese, R. W. Harrison, R. A. Lofthouse, D. Georgakopoulos, D. E. Berkowitz and J. M. Hare, beta(3)-adrenoceptor deficiency blocks nitric oxide-dependent inhibition of myocardial contractility, J. Clin. Invest. 106 (2000), pp. 697-703.

Wang G, Hamid T, Keith R J, et al. Cardioprotective and antiapoptotic effects of heme oxygenase-1 in the failing heart. Circulation. May 4 2010; 121(17):1912-1925.

M. B. West, G. Rokosh, D. Obal, M. Velayutham, Y. T. Xuan and B. G. Hill et al., Cardiac myocyte-specific expression of inducible nitric oxide synthase protects against ischemia/reperfusion injury by preventing mitochondrial permeability transition, Circulation 118 (2008), pp. 1970-1978.

Zhao Q, Wu T G, Jiang Z F, et al. Effect of beta-blockers on beta3-adrenoceptor expression in chronic heart failure. Cardiovasc. Drugs Ther. 2007; 21:85-90.

We claim:

1. A method for treating cardiac hypertrophy or heart failure comprising the step of administering a therapeutically effective amount of a β3 adrenoreceptor agonist to a patient diagnosed with cardiac hypertrophy.

2. The method of claim 1, wherein the β3 adrenoreceptor agonist is selected from the group consisting of BRL 26830A, SR-58611A (Amibegron), GW-427,353 (Solabegron), L-796,568, CL-316,243, LY-368,842, TAK-677, Ro40-2148, ICI D7114, Carvedilol, and Nebivolol.

3. The method of claim 1 further comprising administering to the patient a second therapeutic agent.

4. The method of claim 3, wherein the second therapeutic agent is selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

5. A method for treating cardiac hypertrophy or heart failure comprising the steps of:
a. identifying a patient having cardiac hypertrophy; and
b. administering to the patient a therapeutically effective amount of β3 adrenoreceptor agonist.

6. The method of claim 5, wherein the β3 adrenoreceptor agonist is selected from the group consisting of BRL 26830A, SR-58611A (Amibegron), GW-427,353 (Solabegron), L-796,568, CL-316,243, LY-368,842, TAK-677, Ro40-2148, ICI D7114, Carvedilol, and Nebivolol.

7. The method of claim 5 further comprising administering to the patient a second therapeutic agent.

8. The method of claim 7, wherein the second therapeutic agent is selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

9. A method for treating a cardiovascular disease or condition associated with cardiac hypertrophy comprising the step of administering a therapeutically effective amount of a β3 adrenoreceptor agonist to a patient diagnosed with cardiac hypertrophy.

10. The method of claim 9, wherein said cardiovascular disease or condition associated with cardiac hypertrophy is selected from the group consisting of hypertension, cardiomyopathy, hypertrophic cardiomyopathy, diabetes, systolic heart failure, and non-systolic heart failure.

11. The method of claim 9, wherein the β3 adrenoreceptor agonist is selected from the group consisting of BRL 26830A, SR-58611A (Amibegron), GW-427,353 (Solabegron), L-796,568, CL-316,243, LY-368,842, TAK-677, Ro40-2148, ICI D7114, Carvedilol, and Nebivolol.

12. The method of claim 9 further comprising administering to the patient a second therapeutic agent.

13. The method of claim 12, wherein the second therapeutic agent is selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

14. A method for treating cardiac hypertrophy or heart failure comprising the step of administering a therapeutically effective amount of the β3 adrenoreceptor agonist BRL 26830A to a patient diagnosed with cardiac hypertrophy.
